SEQUENCE LISTING (28 sequences)

SEQ ID NO: 1 - protein, 1278 aa, synthetic construct
  REGION 1..1278: note = gD-SECTO amino acid sequence
MGGAAARLGAVILFVVIVGLHGVRGQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPMGLIAGAVGGSLLAALVICGIVYWMRRRTQKAPKRIRLPHIREDDQPSSHQPLFY

SEQ ID NO: 2 - protein, 1359 aa, synthetic construct
  REGION 1..1359: note = gB-SECTO amino acid sequence
MRQGAPARGCRWFVVWALLGLTLGVLVASAAQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 3 - protein, 640 aa, synthetic construct
  REGION 1..640: note = gC-SRBD amino acid sequence
MAPGRVGLAVVLWSLLWLGAGVSGGSETASTGPTITAGAVTNASEAPTSGSPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFLVLPRPTITMEFGVRHVVCTAGCVPEGVTFAWFLGDDPSPAAKSAVTAQESCDHPGLATVRSTLPISYDYSEYICRLTGYPAGIPVLEHHGSHQPPPRDPTERQVIEAIEWVGIGIGVLAAGVLVVTAIVYVVRTSQSRQRHRR

SEQ ID NO: 4 - protein, 698 aa, synthetic construct
  REGION 1..698: note = gE-SNTD amino acid sequence
  SITE 71: note = X can be any naturally occurring amino acid
MDRGAVVGFLLGVCVVSCLAGTPKTSWRRVSVGEDVSLLPAPGPTGRGPTQKLLWAVEPLDGCGPLHPSWXSLMPPKQVPETVVDAACMRAPVPLAMAYAPPAPSATGGLRTDFVWQERAAVVNRSLVIYGVRETDSGLYTLSVGDIKDPARQVASVVLVVQPAPVPTPPPTPADYDEDDNDEGEGEDESLAGTPASGTPRLPPPPAPPRSWPSAPEVSHVRGVTVRMETPEAILFQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSYRNAVVEQPLPQRGADLAEPTHPHVGAPPHAPPTHGALRLGAVMGAALLLSVLGLSVWACMTCWRRRAWRAVKSRASGKGPTYIRVADSELYADWSSDSEGERDQVPWLAPPERPDSPSTNGSGFEILSPTAPSVYPRSDGHQSRRQLTTFGSGRPDRRYSQASDSSVFW

SEQ ID NO: 5 - DNA, 31 bp, synthetic construct
  misc_feature 1..31: note = PCR primer
  source 1..31: mol_type = other DNA
gaagatctaatatttttattgcaactccctg

SEQ ID NO: 6 - DNA, 31 bp, synthetic construct
  misc_feature 1..31: note = PCR Primer
  source 1..31: mol_type = other DNA
ctagctagcttataaaaggcgcgtcccgtgg

SEQ ID NO: 7 - DNA, 25 bp, synthetic construct
  misc_feature 1..25: note = PCR Primer
  source 1..25: mol_type = other DNA
gctctagattgcgacgccccggctc

SEQ ID NO: 8 - DNA, 35 bp, synthetic construct
  misc_feature 1..35: note = PCR Primer
  source 1..35: mol_type = other DNA
ccttaattaaggttaccaccctgtagccccgatgt

SEQ ID NO: 9 - DNA, 28 bp, synthetic construct
  misc_feature 1..28: note = PCR Primer
  source 1..28: mol_type = other DNA
atcccgagccggggcgtcgcgatgccga

SEQ ID NO: 10 - DNA, 36 bp, synthetic construct
  misc_feature 1..36: note = PCR Primer
  source 1..36: mol_type = other DNA
cgccgatgcggggcgatcctccggggatacggctgc

SEQ ID NO: 11 - DNA, 32 bp, synthetic construct
  misc_feature 1..32: note = PCR Primer
  source 1..32: mol_type = other DNA
cgggccgggacggggcggggcgcttgcgaaac

SEQ ID NO: 12 - DNA, 34 bp, synthetic construct
  misc_feature 1..34: note = PCR Primer
  source 1..34: mol_type = other DNA
aacgcccgccgcgcgcgcgcacgccgcccggacc

SEQ ID NO: 13 - DNA, 32 bp, synthetic construct
  misc_feature 1..32: note = PCR Primer
  source 1..32: mol_type = other DNA
cgccgatgcggggcgatcctccggggatacgg

SEQ ID NO: 14 - DNA, 32 bp, synthetic construct
  misc_feature 1..32: note = PCR Primer
  source 1..32: mol_type = other DNA
tcccgagccggggcgtcgcgatgccgacgccg

SEQ ID NO: 15 - DNA, 32 bp, synthetic construct
  misc_feature 1..32: note = PCR Primer
  source 1..32: mol_type = other DNA
atgagccagacccaacccccggccccagttgg

SEQ ID NO: 16 - DNA, 31 bp, synthetic construct
  misc_feature 1..31: note = PCR Primer
  source 1..31: mol_type = other DNA
cagaaaatgtaaccatacccaaaccgactct

SEQ ID NO: 17 - DNA, 30 bp, synthetic construct
  misc_feature 1..30: note = PCR Primer
  source 1..30: mol_type = other DNA
gggccaccgtcccccccgacaccccaacga

SEQ ID NO: 18 - DNA, 31 bp, synthetic construct
  misc_feature 1..31: note = PCR Primer
  source 1..31: mol_type = other DNA
gtggggctggagggtcagagacggggggcgg

SEQ ID NO: 19 - DNA, 30 bp, synthetic construct
  misc_feature 1..30: note = PCR Primer
  source 1..30: mol_type = other DNA
ctggtgctgccgcggccaaccatcaccatg

SEQ ID NO: 20 - DNA, 30 bp, synthetic construct
  misc_feature 1..30: note = PCR Primer
  source 1..30: mol_type = other DNA
acgcctccacccgtgctgccgtcgctagac

SEQ ID NO: 21 - DNA, 29 bp, synthetic construct
  misc_feature 1..29: note = PCR Primer
  source 1..29: mol_type = other DNA
aagcatcgaccacacccttccccacggga

SEQ ID NO: 22 - DNA, 32 bp, synthetic construct
  misc_feature 1..32: note = PCR Primer
  source 1..32: mol_type = other DNA
aaacaggatagcttccggagtctccatacgca

SEQ ID NO: 23 - DNA, 30 bp, synthetic construct
  misc_feature 1..30: note = PCR Primer
  source 1..30: mol_type = other DNA
taccggaacgcggtggtggaacagcccctc

SEQ ID NO: 24 - DNA, 30 bp, synthetic construct
  misc_feature 1..30: note = PCR Primer
  source 1..30: mol_type = other DNA
aaaaatcaaccgggagacaacattgccaat

SEQ ID NO: 25 - DNA, 28 bp, synthetic construct
  misc_feature 1..28: note = PCR Primer
  source 1..28: mol_type = other DNA
atcccgagccggggcgtcgcgatgccga

SEQ ID NO: 26 - DNA, 36 bp, synthetic construct; identical to SEQ ID NO: 10
  misc_feature 1..36: note = PCR Primer
  source 1..36: mol_type = other DNA

SEQ ID NO: 27 - DNA, 32 bp, synthetic construct; identical to SEQ ID NO: 11
  misc_feature 1..32: note = PCR Primer
  source 1..32: mol_type = other DNA

SEQ ID NO: 28 - DNA, 34 bp, synthetic construct; identical to SEQ ID NO: 12
  misc_feature 1..34: note = PCR Primer
  source 1..34: mol_type = other DNA

(19) United States
(12) Patent Application Publication
(10) Pub. No.: US 2026/0335432 A1
(43) Pub. Date: Oct. 29, 2026

(54) REPLICATION INCOMPETENT HERPES SIMPLEX VIRUS TYPE 1 VIRAL VACCINE

(71) Applicant: IMMVIRA BIOPHARMACEUTICALS CO., LIMITED, Suzhou, Jiangsu (CN)

(72) Inventors: Yuanyuan LIU, Suzhou, Jiangsu (CN); Yonghong LIU, Suzhou, Jiangsu (CN); Tianyi DENG, Suzhou, Jiangsu (CN); Yuyun ZENG, Suzhou, Jiangsu (CN); Xiaoqing CHEN, Suzhou, Jiangsu (CN); Grace Guoying ZHOU, Suzhou, Jiangsu (CN)

(21) Appl. No.: 19/101,316

(22) PCT Filed: Aug. 4, 2023
(86) PCT No.: PCT/CN2023/111125

§ 371 Date: Feb. 5, 2025

(30) Foreign Application Priority Data
Aug. 5, 2022 (WO) PCT/CN2022/110565

(51) Int. Cl. A61K 39/245; A61K 39/00; A61K 39/215; A61K 39/385

(52) U.S. Cl. CPC A61K 39/245; A61K 39/215; A61K 39/385; A61K 2039/5254; A61K 2039/6075; A61K 2039/70

(57) ABSTRACT

Disclosed is a replication incompetent HSV-1 viral vaccine comprising a modified genome of HSV-1 and at least one antigen. The modification comprises a deletion of internal repeats, an inactivating mutation in ICP47 and an inactivating mutation in the other copy of ICP4. A first antigen of the at least one antigen is driven by a promoter of an immediate early gene, such as ICP4. In a specific example, the HSV-1 viral vaccine expresses antigens from SARS-Cov, SARS-Cov-2 and variants thereof and is used for inducing immune responses against sarbecoviruses in a subject to which the vaccine is administered.

### CROSS REFERENCE TO RELATED APPLICATIONS

This application is a U.S. National Stage Application of International Application No. PCT/CN2023/111125, filed on Aug. 4, 2023, which claims priority to International Application No. PCT/CN2022/110565, filed Aug. 5, 2022. The contents of each of which are hereby incorporated by reference in their entirety.

### REFERENCE TO AN ELECTRONIC SEQUENCE LISTING

The contents of the electronic sequence listing (395170.xml; Size: 40,768 bytes; and Date of Creation: Feb. 4, 2025) is herein incorporated by reference in its entirety.

### TECHNICAL FIELD

The present disclosure relates to a replication incompetent herpes simplex virus type 1 (HSV-1) vaccine delivering at least one antigen from non-HSV-1 microorganism, e.g., virus, bacterium or parasite, and in particular, to a replication incompetent HSV-1 viral vaccine expressing domains of Spike glycoprotein from SARS-Cov, SARS-Cov-2 and variants thereof. The present disclosure also relates to a vaccine composition comprising the viral vaccine and a pharmaceutically acceptable carrier. The present disclosure further relates to a method for inducing immune response against sarbecovirus in a subject.

### BACKGROUND

Coronavirus 19 Disease (COVID-19) is caused by the severe acute respiratory syndrome coronavirus type 2 (SARS-Cov-2) or variants thereof, whose infection in humans causes mild or severe clinical manifestations that mainly affect the respiratory system. SARS-Cov-2 contains the Spike(S) glycoprotein on its surface, which is the main target for current vaccine development because antibodies directed against this protein can neutralize the infection. Companies and academic institutions have developed vaccines based on the S glycoprotein, as well as its antigenic domains and epitopes, which have been proven effective in generating neutralizing antibodies. However, the emergence of new SARS-Cov-2 variants could affect the effectiveness of vaccines. A pan-sarbecovirus vaccine would be desirable in view of the ongoing evolution of the virus.

### SUMMARY

A first aspect of the present disclosure is related to a replication incompetent Herpes Simplex Virus type 1 (HSV-1) viral vaccine, comprising a modified genome of HSV-1 and at least one antigen, wherein the modification comprises a deletion of an internal inverted repeat region which causes deletions of one copy of each of double-copy genes including ICP0, ICP34.5, ICP4 and latency-associated transcript (LAT), an inactivating mutation in ICP47 and an inactivating mutation in the other copy of ICP4 in a terminal repeat, and wherein a first antigen of the at least one antigen is driven by a promoter of an immediate early gene of wild-type HSV-1.

In some embodiments, the immediate early gene of wild-type HSV-1 is ICP0, ICP27, ICP4, ICP22 or ICP47. In some embodiments, the immediate early gene of wild-type HSV-1 is ICP4.

In some embodiments, the inactivating mutation in the other copy of ICP4 in the terminal repeat is a deletion in a coding sequence of ICP4. In some of the embodiments where a deletion occurs in a coding sequence of ICP4, the first antigen of the at least one antigen is operably linked to the promoter of ICP4 in the terminal repeat.

In some embodiments, the first antigen of the at least one antigen together with the driving promoter is inserted into a position corresponds to the deleted internal inverted repeat region.

In any of the above embodiments, the replication incompetent HSV-1 viral vaccine further comprises a second antigen that is fused into a first HSV-1 glycoprotein.

In any of the above embodiments, the replication incompetent HSV-1 viral vaccine further comprises a third antigen that is fused into a second HSV-1 glycoprotein.

In some embodiments, the first or second HSV-1 glycoprotein is glycoprotein gC or gE.

In some embodiments, the glycoprotein gC is altered to inactivate C3 binding, and wherein the glycoprotein gE is altered to inactivate FcR binding. In some embodiments, the glycoprotein gC contains a deletion in C3 binding domain, and wherein the glycoprotein gE contains a deletion in FcR binding domain.

In some embodiments, the first antigen is linked to a glycoprotein gB or gD signal peptide at N terminus, and to a transmembrane-intravirion domain of glycoprotein gB or gD at C terminus.

In some embodiments, the first, second or third antigen is from a virus, a bacterium or a parasite. In some embodiments, the first, second and third antigen are from sarbecovirus. In some embodiments, the first, second and third antigen are from SARS-Cov, SARS-Cov-2 and variants thereof. In some embodiments, the first antigen is from delta or omicron variant of SARS-Cov-2, the second and third antigens are from SARS-Cov, SARS-Cov-2 and variants thereof. In some embodiments, the first antigen is from delta variant of SARS-Cov-2, the second antigen is from SARS-Cov Tor2 strain, and the third antigen is from SARS-Cov-2 Wuhan-Hu-1 strain.

In some embodiments, the first antigen is an ectodomain of Spike glycoprotein of delta variant of SARS-Cov-2 or an immunogenically equivalent variant thereof. In some embodiments, the ectodomain or the immunogenically equivalent variant thereof is linked to a glycoprotein gB signal peptide at N terminus, and to a transmembrane-intravirion domain of glycoprotein gB at C terminus. In some embodiments, the immunogenically equivalent variant of the ectodomain has a K986P/V987P mutation and/or a 682-GSAS-685 mutation.

In some embodiments, one of the second and third antigen is a receptor binding domain of Spike glycoprotein of SARS-Cov Tor2 strain, and the other is an N-terminal domain of Spike glycoprotein of SARS-Cov-2 Wuhan-Hu-1 strain, or an immunogenically equivalent variant each thereof.

In some embodiments, the second antigen is a receptor binding domain of Spike glycoprotein of SARS-Cov Tor2 strain or an immunogenically equivalent variant thereof and the first HSV-1 glycoprotein is glycoprotein gC. In some embodiments, the receptor binding domain or an immunogenically equivalent variant thereof is fused into glycoprotein gC in replace of its C3 binding domain.

In some embodiments, the second antigen is an N-terminal domain of Spike glycoprotein of SARS-Cov-2 Wuhan-Hu-1 strain or an immunogenically equivalent variant thereof and the first HSV-1 glycoprotein is glycoprotein gE. In some embodiments, N-terminal domain of Spike glycoprotein of SARS-Cov-2 Wuhan-Hu-1 strain or an immunogenically equivalent variant thereof is fused into glycoprotein gE in replace of its FcR binding domain.

In some embodiments, the inactivating mutation in ICP47 is a deletion in a coding sequence of ICP47.

In some embodiments, the inverted internal repeat region is replaced by a promoter (such as a CMV, EF1α, CAG, or UbC promoter) followed by three repeats of stop codon.

In some embodiments, the modified genome contains one copy of ICP0, LAT and ICP34.5, UL1 to UL56, and US1 to US11.

Another aspect of the disclosure relates to a vaccine composition, comprising a replication incompetent HSV-1 viral vaccine disclosed herein, and a pharmaceutically acceptable carrier.

A further aspect of the disclosure relates to a method of inducing immune response in a subject, comprising administering to the subject a pharmaceutically effectively amount of the viral vaccine disclosed herein.

### BRIEF DESCRIPTION OF THE DRAWINGS

FIG. 1 shows schematic diagrams of the HSV-1 (F), MVR-ΔIR4 and MVR-ΔIR47 genomes.

FIG. 2 shows MVR-ΔIR4 and MVR-ΔIR47 viruses can only replicate in the ICP4 complementing cell line E5.

FIG. 3 shows construction model of non-replicating HSV-1 based sarbecovirus spike protein vaccine virus.

FIG. 4 shows schematic diagram of the MVR-ΔIR47, MVR-S-gB or MVR-S-gD genomes.

FIG. 5 shows the structure of gC-S^{RBD} chimera, in which the gC protein amino acids from 275-367 was replaced by the RBD domain of SARS coronavirus Tor2 (SARS Tor2) spike.

FIG. 6 shows the structure of gE-S^{NTD} chimera, in which the gE protein amino acids from 237-382 was replaced by the NTD domain of SARS-Cov-2 WT Wuhan-Hu-1 spike.

FIG. 7 shows the structure of gB-S^{ECTO} chimera, in which the S^{ECTO} was connected to the transmembrane domain and cytoplasmic tail (TM/CT) of the gB protein.

FIG. 8 shows the structure of gD-S^{ECTO} chimera, in which in which the S^{ECTO} was connected to the transmembrane domain and cytoplasmic tail (TM/CT) of the gD protein.

FIG. 9 shows the accumulation of gC, gC-S^{RBD}, gE-S^{NTD}, gB-S^{ECTO} or gD-S^{ECTO} proteins in MVR-S-gB and MVR-S-gD infected E5 cells.

FIG. 10 shows the accumulation of HSV-1 representative viral proteins in MVR-S-gB virus infected E5 and Vero cells.

FIG. 11 shows the design of animal immunization.

FIG. 12 shows the microneutralization assays using HIV-based pseudotyped virus. ACE2-Fc represents a positive control which is a fusion protein comprising ACE2 functional domain and human Fc fragment. All the groups vaccinated with MVR-S-gB induced neutralization antibodies against Wuhan-Hu-1 strain (FIG. 12A) and Delta strain (FIG. 12B) in a dose-dependent manner. The groups immunized with 4×10⁶ PFU or 2×10⁷ PFU of MVR-S-gB induced neutralization antibodies against Omicron BA1 strain (FIG. 12C).

FIG. 13 shows MVR-S-gB vaccines can induce long-lasting neutralizing antibodies up to at least 6 months. Serum samples were collected at 3 months (FIG. 13A) and 6 months (FIG. 13B) after third vaccination and used for pseudotyped virus neutralization assays.

### DETAILED DESCRIPTION

### Definitions

The term “antigen” is used herein to mean molecules that trigger the production of antibodies by inducing an immune response. Typical antigens are proteins, or ectodomains or a part thereof, present on the surface of pathogens, such as bacteria, fungi, viruses, and other foreign particles. When these harmful agents enter the body, it induces an immune response in the body for the production of antibodies. The “antigen” contained in the viral vaccine of present disclosure is not intended to include an HSV-1 antigen. That is, the antigen of the present disclosure is a non-HSV-1 protein, an ectodomain, or a fragment each thereof.

By “inactivating mutation” is meant any mutation in the genomic DNA sequence of a target gene that results in the target gene inactive, non-functional, or absent, including but not limited to insertions, deletions, or substitutions of one or more nucleic acids in the genomic DNA sequence of the target gene, in particular, the coding sequence of the gene of interest. In some embodiments, the inactivating mutation reduces or eliminates mRNA transcription, thereby reducing or eliminating the expression level of the encoded mRNA transcript and protein. In some embodiments, the inactivating mutation reduces or inhibits mRNA translation, thereby reducing the expression level of the encoded protein. In some embodiments, the inactivating mutation encodes a modified protein with reduced or altered function compared to the unmodified (i.e., wild-type) version of the protein. For example, an inactivating mutation in ICP47 may include a deletion of the coding sequence (CDS) of the ICP47 gene, or a substitution of one or more nucleic acids in the CDS of the ICP47 gene that leads to the production of a non-functional ICP47 protein. For example, an inactivating mutation in ICP4 may include a deletion of the coding sequence (CDS) of the ICP4 gene, or a substitution of one or more nucleic acids in the CDS of the IC4 gene that leads to the production of a non-functional ICP4 protein.

The term “a deletion in a coding sequence” is meant that a deletion of a fragment of the coding sequence or a deletion of the whole coding sequence. In some embodiments, a deletion in a coding sequence results in the deletion of the coding sequence. In some embodiments, a deletion in a coding sequence does not result in the deletion of the regulatory elements (such as a promoter sequence, an enhancer sequence, a ribosome binding site, or a transcription terminator) of the corresponding gene.

By “fused” is meant that the components (e.g., an NTD domain, a RBD domain, and a signal peptide) are linked by peptide bonds, either directly or via one or more peptide linkers. The term “fused into” is meant that a smaller component (e.g., an NTD domain) is linked to a larger component (e.g., an ectodomain of an HSV-1 glycoprotein, such as gC or gE) by peptide bonds at one terminus (N or C terminus) or any position therebetween of the latter. In some embodiments, an intermediate fragment of the larger component is replaced by the small component.

By “signal peptide” of a glycoprotein (such as gB or gD) it is meant the signal peptide of the respective HSV-1 glycoprotein, either as naturally occurring or having one or more conservative mutations compared to the parent sequence.

By “transmembrane-intravirion domain” it is meant that the transmembrane and intravirion domains (intravirion domain being also referred to herein as cytoplasmic tail) are directly linked to each other as naturally occurring, or indirectly linked to each other by a suitable peptide linker.

“HSV-1 glycoprotein” as used herein is meant the 12-13 virally encoded glycoproteins on the HSV-1 viral envelope which help the virus to interact with target cells. Of the 12 or more glycoproteins present on the HSV-1 viral envelope, coordinated action of five glycoproteins: gC, gD, gB and the heterodimer gH and gL, is required for viral entry into the target cell. gC and gB independently interact with cell surface heparan sulphate proteoglycan and mediate initial viral binding. In the absence of both gB and gC, virus binding to the cell surface is severely reduced.

Glycoprotein gB is the most conserved entry glycoprotein among herpesviruses with an amino acid sequence identity of around 50% within each subfamily. gB is a type I transmembrane protein consisting of a signal peptide, a large ectodomain, a single-spanning TM, a membrane proximal region (MPR), and a long CTD comprising more than 90 residues. gB is the bona fide herpesvirus fusion protein. The earliest evidence pointing to its involvement in membrane fusion came from the observation that syncytial HSV-1 strains harbored mutations in the gB gene. The first crystal structure of gB from HSV-1 provided the most direct evidence that it is indeed the bona fide protein fusogen of herpesviruses. Despite a lack of sequence conservation and having significantly different sizes (~700 vs ~400 aa), the HSV-1 gB ectodomain was shown to have a conserved secondary, tertiary and quaternary structure with the fusion protein G of the otherwise unrelated vesicular stomatitis virus (VSV). So far, structures of gB ectodomains from alphaherpesviruses HSV-1 and PrV, the betaherpesvirus human cytomegalovirus (HCMV) and the gammaherpesvirus EBV have been determined, revealing a highly conserved fold. A full length of the gB amino acid sequence is available from UniProtKB/Swiss-Prot: P06437.2 (KOS strain), UniProtKB/Swiss-Prot: P06436.1 (F strain), UniProtKB/Swiss-Prot: P10211.1 (17 strain) and etc.

An exemplary sequence of the signal peptide of gB is aa 1 to aa 30 of UniProtKB/Swiss-Prot: P06437.2, aa 1 to aa 29 of UniProtKB/Swiss-Prot: P06436.1, or aa 1 to aa 30 of UniProtKB/Swiss-Prot: P10211.1.

An exemplary transmembrane-intravirion domain of gB is aa 775 to aa 904 of UniProtKB/Swiss-Prot: P06437.2, aa 774 to aa 903 of UniProtKB/Swiss-Prot: P06436.1, or aa 775 to aa 904 of UniProtKB/Swiss-Prot: P10211.1.

Glycoprotein gD is the main receptor-binding glycoprotein and binds to three classes of cell receptor: (1) herpes viral entry mediator (HVEM); a member of the TNF receptor family; (2) nectin 1 and nectin 2; members of the immunoglobulin superfamily and (3) 3-O sulphated heparan sulphate. The binding of gD to one of these receptors initiates the conformational changes mediated by gB, gD, gH, and gL and triggers the fusion between the virion envelope and the plasma membrane. A full length of the gD amino acid sequence is available from UniProtKB/Swiss-Prot: P57083.1 (Patton strain), UniProtKB/Swiss-Prot: Q05059.1 (F strain), UniProtKB/Swiss-Prot: A1Z0Q5.2 (KOS strain), UniProtKB/Swiss-Prot: Q69091.1 (strain 17) and etc.

An exemplary sequence of the signal peptide of gD is aa 1 to aa 25 of UniProtKB/Swiss-Prot: P57083.1, aa 1 to aa 25 of UniProtKB/Swiss-Prot: Q05059.1, aa 1 to aa 25 of UniProtKB/Swiss-Prot: A1Z0Q5.2, or aa 1 to aa 25 of UniProtKB/Swiss-Prot: Q69091.1.

An exemplary transmembrane-intravirion domain of gD is aa 340 to aa 394 of UniProtKB/Swiss-Prot: P57083.1, aa 341 to aa 394 of UniProtKB/Swiss-Prot: Q05059.1, aa 341 to aa 394 of UniProtKB/Swiss-Prot: A1Z0Q5.2, or aa 341 to aa 394 of UniProtKB/Swiss-Prot: Q69091.1.

Glycoprotein gE functions as a receptor for the Fc portion of immunoglobulin G (IgG) (FcγR) and plays a role in virus spread from cell to cell. gE interacts with glycoprotein gI to form a noncovalent heterodimer complex that increases Fc binding affinity so that the gE-gI complex binds IgG monomers, whereas gE alone binds IgG aggregates but not monomers. Functions assigned to the IgG Fc domain include activation of the classical complement pathway and binding to immune effector cells that express FcγRs. In vitro studies to address the functions of the HSV-1 FcγR have demonstrated that the FcγR protects the virus from antibody-dependent complement neutralization, antibody-dependent cellular cytotoxicity (ADCC), and Fc-mediated attachment of granulocytes to HSV-1-infected cells. HSV-1 gE is required for efficient spread of virus from one epithelial cell to another and from epithelial cells to neurons. HSV-1 gE also mediates targeting of capsid, tegument, and viral glycoproteins from the neuron cell body into axons. Partially overlapping gE domains mediate FcγR activity and spread, posing a challenge to separate these functions. The complete amino acid sequences of gE of different HSV-1 strains are available from GenBank ADD60055.1 (F strain), UniProtKB/Swiss-Prot: P04488.1 (17 strain), GenBank: AFE62896.1 (KOS strain) and etc.

Residues 235 to 380 of wild-type gE form a continuous IgG aggregate binding domain and are essential for FcR activity. By “FcR binding domain” of gE, it is meant a region between amino acid 235 and 380 or the region plus adjoining sequences (such as one or more, e.g., 1 to 10, residues at its N or C termini), in the case of HSV-1 F strain (GenBank ADD60055.1). By “a deletion in FcR binding domain” it is meant that a fragment or the full length of the FcR binding domain is deleted. For example, a fragment of aa 280 to aa 286, or aa 299 to aa 306, located within the FcR binding domain is deleted. Correspondingly, the term “inactivate FcR binding” is meant to disenable the FcR binding function of the gE glycoprotein. This can be achieved by a deletion in the FcR binding domain, or an insertion or substitution of one or more nucleotides in the coding sequence of the FcR binding domain.

HSV-1 glycoprotein C binds complement component C3b and inhibits the interaction of C5 and properdin (P) with C3b, blocking activation of both the classical and alternative complement pathways. HSV-1 gC prevents complement-mediated neutralization of cell-free virus, inhibits complement-mediated lysis of infected cells, and contributes to virulence in vivo, as viruses deficient in binding C3b or blocking C5 and P from interacting with C3b are more attenuated than wild-type virus in a murine flank model of infection. The complete amino acid sequence of gC of different HSV-1 strains are available from GenBank ADD60042.1 (F strain), GenBank: AKM76368.1 (17+ strain), GenBank: AAA45779.1 (macroplaque strain), GenBank: CAB40083.1 (HSZP strain), GenBank: AFH78104.1 (McKrae strain) and etc.

Wild-type gC has four C3 binding regions, i.e., binding regions I (aa 124-137), II (aa 276-292), III (aa 339-366), and IV (aa 223-246), each of which is required to bind C3. By “C3 binding domain” it is meant any of the four binding regions or combination thereof. That is, a C3 binding domain can be any of the binding region I, the binding region II, the binding region III, the binding region IV, the binding regions II/III, the binding regions IV/II, the binding regions II/III/IV, the binding regions I/IV, the binding regions I/IV/II, or the binding regions I/II/III/IV. When two or more binding regions are designated, it is meant to include amino acids between the two or more binding regions. For example, binding regions II/III are meant to include the binding regions II, III, and amino acids 293 to 338 between the two regions. Thus, a deletion in the C3 binding domain or a grammatical variant thereof is meant that a deletion of any of the regions I to IV or combination thereof. For example, a deletion in the C3 binding domain can be a deletion of the binding region I, II, III, or IV. Alternatively, a deletion in the C3 binding domain can be a deletion of binding regions II/III, IV/II, I/IV, IV/II/III, or I/IV/II/III. The deletion occurs in the region of aa 124 to aa 366 of the wild-type gC. In a preferable example, the deletion in the C3 binding domain is a deletion of the binding regions II/III, i.e., aa 276 to aa 366. Correspondingly, the term “inactivate C3 binding” is meant a manipulation of the genome to inactivate any of the binding regions I to IV, or their combination. The manipulation can be insertion, deletion, or substitution of one or more nucleotides in the coding sequences of the binding regions I to IV.

The term “sarbecovirus” is a viral subgenus containing SARS-Cov, SARS-Cov-2 and various variants. See Schoch C L, et al. NCBI Taxonomy: a comprehensive update on curation, resources and tools. Database (Oxford). 2020: baaa062. PubMed: 32761142 PMC: PMC7408187 (see also https://www.ncbi.nlm.nih.gov/Taxonomy/Browser/wwwtax.cgi?mode=Undef&id=25 09511&lvl=3&lin=f, last accessed on 20 Jul. 2022). Sarbecoviruses SARS and SARS-Cov-2 can cause severe pulmonary disease and each have diarrhea and fecal shedding as a prominent feature. In each case, comorbidities and age increase the risk of severe disease. SARS-Cov-2 also infects the upper respiratory tract and overall has lower mortality than SARS-Cov (though if disease is severe, mortality is similar to that of SARS). Long COVID is seen with SARS-Cov-2. Goals for vaccination should include prevention of severe disease and long COVID. Increasing numbers of coronaviruses are being identified and the substantial genetic diversity among them increases the difficulty of developing vaccines with broad immunity to these viruses. Climate change may alter diversity and likelihood of crossover to humans of additional sarbecoviruses, most of which have bat hosts. Completely new coronaviruses could emerge in humans due to mutations or due to changes in contact between humans & infected vectors or host (which can be influenced by weather and changing opportunities for animal exposure). Animal reservoirs present challenges and opportunities for control.

A “pan-sarbecovirus vaccine” is a vaccine that can induce immune response against more than one sarbecovirus or its variants in a subject that is administered with the vaccine. An aim of the present disclosure is to provide a pan-sarbecovirus vaccine by delivering antigens from more than one sarbecovirus using a single replication incompetent HSV-1 vector. It is contemplated that the HSV-1 vector can also be used as a vaccine platform for delivering a single antigen, or antigens from viruses different from sarbecovirus.

Severe acute respiratory syndrome (SARS) was the first new infectious disease identified in the twenty-first century. A global effort coordinated by WHO led to the identification, in April 2003, of a new coronavirus, SARS-coronavirus (SARS-Cov, also known as SARS-Cov-1, or SARS coronavirus), as the agent that caused the outbreak. SARS-Cov is an enveloped, single and positive-stranded RNA virus. Its genome RNA encodes a non-structural replicase polyprotein and structural proteins, including spike(S), envelope (E), membrane (M) and nucleocapsid (N) proteins. Neutralizing antibodies and/or T-cell immune responses can be raised directly against several SARS-Cov proteins, but mainly target the S protein, suggesting that S protein-induced specific immune responses play important parts in the fight against SARS-Cov infection. The spikes of SARS-Cov are composed of trimers of S protein. The SARS-Cov S protein encodes a surface glycoprotein precursor that is predicted to be 1,255 amino acids in length, and the amino terminus and most of the protein is predicted to be on the outside of the cell surface or the virus particles. The predicted S protein consists of a signal peptide (amino acids 1-12) located at the N terminus, an extracellular domain (amino acids 13-1,195), a transmembrane domain (amino acids 1,196-1,215) and an intracellular domain (amino acids 1,216-1,255). Similar to other coronaviruses, the S protein of SARS-Cov can be cleaved into the S1 and S2 subunits by proteases, such as trypsin, factor Xa and cathepsin L. Angiotensin-converting enzyme 2 (ACE2) has been identified as the receptor of SARS-Cov. A fragment that is located in the S1 subunit and spans amino acids 318-510 is the minimal receptor-binding domain (RBD). Crystallographic studies have shown the structure of RBD complexed with its receptor ACE2.

SARS-Cov S protein has pivotal roles in viral infection and pathogenesis. S1 recognizes and binds to host receptors, and subsequent conformational changes in S2 facilitate fusion between the viral envelope and the host cell membrane. The RBD in S1 is responsible for virus binding to host cell receptors. K341 of ACE2 and R⁴⁵³ of the RBD are important for the complex formation. N479 and T487 of the RBD are important for the high-affinity association of S protein with ACE2. An exemplary amino acid sequence of the RBD domain of SARS-Cov is available from NCBI Reference Sequence: YP_009825051.1 at aa 306 to aa 527.

Coronavirus 19 Disease (COVID-19) is caused by the severe acute respiratory syndrome coronavirus type 2 (SARS-Cov-2), whose infection in humans causes mild or severe clinical manifestations that mainly affect the respiratory system. SARS-Cov-2 contains the Spike ((also referred to herein as Spike glycoprotein, “S”, S glycoprotein or spike) glycoprotein on its surface, which is the main target for current vaccine development because antibodies directed against this protein can neutralize the infection. This protein is responsible for anchoring to the host receptor, the angiotensin-converting enzyme 2 (ACE2).

S glycoprotein is responsible for the entry of the virus into host cells, where it begins to spread, but it can also be recognized by the immune system triggering a protective response, the main objective of vaccines. The viral S glycoprotein is in a metastable prefusion state, through the association of subunits 1 and 2 (S1 and S2) via noncovalent interactions. The S1 subunit of S is made up of 672 amino acids (residues 14-685) and contains four domains: an N-terminal domain (NTD), the receptor binding domain (RBD), and the subdomains 1 and 2 (SD1 and SD2). RBD has received more attention because it is recognized as the intermediary factor in the virus-host cell interaction, through the interaction of its receptor-binding motif (RBM) with the angiotensin converting enzyme 2 (ACE2) of the host cell. The binding of RBM to the ACE2 receptor is crucial in the viral infection process, since it has been shown that this interaction induces the transition of S from a metastable prefusion state to a more stable post-fusion state, which is required for membrane fusion between the virus and the host cell. The S2 subunit is composed of 588 amino acids (residues 686-1273), contains an N-terminal fusion peptide (FP) and two heptad repeats (HR1 and HR2) that mediate the association of the S2 subunit to the host membrane. The S2 subunit also has a transmembrane domain (TM) and an intravirion tail which serves to attach the S glycoprotein to the virus membrane.

An exemplary NTD amino acid sequence of the S glycoprotein of SARS-Cov-2 is available from UniProtKB/Swiss-Prot: PODTC2.1 at aa 13 to aa 304. An exemplary RBD amino acid sequence of the S glycoprotein of SARS-Cov-2 is available from UniProtKB/Swiss-Prot: PODTC2.1 at aa 319 to aa 541.

As used herein, the terms “replication incompetent”, “replication deficient” or “non-replicating” have their ordinary meaning, such as, a virus that is propagation incompetent as a result of modifications to its genome. Thus, once such recombinant virus infects a cell, the only course it can follow is to express any viral and heterologous protein contained in its genome. In a specific embodiment, the replication defective virus provided herein may contain genes encoding nonstructural proteins and are self-sufficient for RNA transcription and gene expression. However, these vectors lack genes encoding structural proteins, so that a helper genome is needed to allow them to be packaged into infectious particles. In the present disclosure, the replication incompetent virus lacks both copies of ICP4. This could be achieved, for example, by deletions of the internal inverted repeats in which one of the copies of ICP4 resides and the coding sequence of the other copy of ICP4 in a terminal repeat.

As used herein, the term “immunogenically equivalent variant” means a variant having one or more conservative mutations compared to the reference antigen but retains the immunogenicity of the reference antigen. The immunogenically equivalent variant retains one or more or all of the epitopes of the reference antigen. In some embodiments, the immunogenically equivalent variant retains all the epitopes of the reference antigen.

By “conservative mutations” it is meant one or more changes of an amino acid residue to a homologous one (such as isoleucine to leucine, aspartate to glutamate, or cysteine to serine), which is not expected to perturb the protein significantly. Furthermore, nucleotide or amino acid substitutions, deletions, or insertions leading to conservative mutations or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

An “adjuvant” is used herein to refer to an ingredient used in a vaccine composition that helps create a stronger immune response in people receiving the vaccine. Adjuvants help the body to produce an immune response strong enough to protect the person from the disease he or she is being vaccinated against. Adjuvanted vaccines can cause more local reactions (such as redness, swelling, and pain at the injection site) and more systemic reactions (such as fever, chills and body aches) than non-adjuvanted vaccines. Aluminum salts, such as aluminum hydroxide, aluminum phosphate, and aluminum potassium sulfate have been used safely in vaccines for more than 70 years. Aluminum-containing adjuvants are vaccine ingredients that have been used in vaccines since the 1930s. Small amounts of aluminum are added to help the body build stronger immunity against the germ in the vaccine. Aluminum is one of the most common metals found in nature and is present in air, food, and water. Scientific research has shown the amount of aluminum exposure in people who follow the recommended vaccine schedule is low and is not readily absorbed by the body. Beginning in 2009, monophosphoryl lipid A (MPL) was used in one U.S. vaccine (Cervarix®); however, the vaccine is no longer available in the United States due to low market demand. This immune-boosting substance was isolated from the surface of bacteria. MF59 is the adjuvant contained in Fluad (an influenza vaccine licensed for adults aged 65 or older). MF59 is an oil-in-water emulsion composed of squalene, which is a naturally occurring oil found in many plant and animal cells, as well as in humans. MF59, used in flu vaccines in Europe since 1997 and in the United States since 2016, has been given to millions of people and has an excellent safety record. AS01B is an adjuvant suspension used with the antigen component of Shingrix vaccine. Shingrix is the recombinant zoster vaccine recommended for persons aged 50 years or older. AS01B is made of up of monophosphoryl lipid A (MPL), an immune-boosting substance isolated from the surface of bacteria, and QS-21, a natural compound extracted from the Chilean soapbark tree (Quillaja saponaria Molina). In pre-licensure clinical trials, AS01B was associated with local and systemic reactions, but the overall safety profile was reassuring. AS01B is also a component of vaccines currently being tested in clinical trials, including malaria and HIV vaccines. To date, these trials have included over 15,000 people. CpG 1018 is a recently developed adjuvant used in Heplisav-B vaccine. It is made up of cytosine phosphoguanine (CpG) motifs, which is a synthetic form of DNA that mimics bacterial and viral genetic material. When CpG 1018 is included in a vaccine, it increases the body's immune response. In pre-licensure clinical trials, adverse events after Heplisav-B were comparable to those observed after another U.S.-licensed, non-adjuvanted hepatitis B vaccine.

As used herein, the terms “antigen” refers to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

### HSV-1 Viral Vaccines

The HSV-1 genome consists of two covalently linked components, designated L and S. Each component consists of unique sequences (UL for the L component, US for the S component) flanked by inverted repeats. The inverted repeats of the L component are designated as ab and b′a′. The inverted repeats of the S component are designated as a′c′ and ca. The inverted repeats regions contain double-copy of transcriptional units. There are at least five open reading frames known in the art that have double copies, the proteins of which are designated ICP0, ICP4, ICP34.5, ORF P and ORF O, respectively. The inverted repeats b′a′ and a′c′ (b′a′-a′c′) are joined to form an internal inverted repeat region or internal inverted repeats. In contrast, the inverted repeats ab and ca are herein referred to as terminal repeat regions or terminal repeats.

An aspect of the present disclosure is directed to a replication incompetent Herpes Simplex Virus type 1 (HSV-1) viral vaccine, comprising a modified genome of HSV-1 and at least one antigen, wherein the modification comprises a deletion of an internal inverted repeat region which causes deletions of one copy of each of double-copy genes including ICP0, ICP34.5, ICP4 and latency-associated transcript (LAT), an inactivating mutation in ICP47 and an inactivating mutation in the other copy of ICP4 in a terminal repeat, and wherein a first antigen of the at least one antigen is driven by a promoter of an immediate early gene of wild-type HSV-1.

In some embodiments, the deletion of the internal inverted repeat region causes the excision of nucleotides 117005 to 132096 in the genome of an HSV-1 F strain, the genome of which is available by GenBank Accession No. GU734771.1. It will be appreciated by a skilled person in the art that the exact starting and ending positions of the nucleotides to be deleted according to the present disclosure depend on the strains and genome isomers of the HSV-1 virus and can be easily determined by known techniques in the art. It should be understood that the present disclosure is not intended to be limited to any specific genome isomers nor strains of an HSV-1 virus. It also will be appreciated by the person skilled in the art that other strains are also possible as long as the genome DNA is sequenced. Sequencing technologies are easily available in literature and on market. For example, in another embodiment, the deletion may be performed on an HSV-1 strain 17, the genome of which is available by GenBank Accession No. NC_001806.2. In another embodiment, the deletion may be performed on a strain KOS 1.1, the genome of which is available by GenBank Accession No. KT899744. It should be noted that a large fraction of the deleted sequence does not encode proteins, but are duplicated non-coding sequences interspaced between the deleted region, e.g., introns of ICP0, LAT domain, “a” sequence and etc. The deletion of the internal inverted repeat region causes the deletions of one copy of each of ICP0, LAT, ICP4, ICP34.5, ORF P, and ORF O.

In some embodiments, the inactivating mutation in ICP47 (also known as US12) is a deletion of the coding sequence of ICP 47. For example, in one embodiment, the deletion of the coding sequence of ICP 47 is a deletion of nucleotides 145152 to 145418 in the genome of an HSV-1 F strain (GenBank: GU734771.1). In some embodiments, the inactivating mutation in ICP47 is a deletion of the gene ICP47. For example, in one embodiment, the deletion of the coding sequence of ICP 47 is a deletion of nucleotides 143988 to 146011 in the genome of an HSV-1 F strain (GenBank: GU734771.1). In some embodiments, the inactivating mutation in ICP47 is a deletion of a fragment of nucleotides overlapping nucleotide sequence 145152 to 145418 but within nucleotides 143988 to 146011 in the genome of an HSV-1 F strain (GenBank: GU734771.1). For example, the deleted sequence may start with any nucleotide after 143988 and ends within 145152 to 145418, or may start with any nucleotide within 145152 to 145418 but ends before 146011 of an HSV-1 F strain (GenBank: GU734771.1). A skilled person in the art would appreciate that when a different strain (e.g., KOS, 17, and etc.) or a different isomer is used, the specific starting and ending positions of the deletion may vary and can be determined according to the sequence information available in GenBank database.

In some embodiments, the inactivating mutation in ICP47 is an insertion, deletion, or substitution of a nucleotide in the coding sequence of the ICP47 that causes a missense mutation giving rise to a non-functional ICP47 protein. In some embodiments, the inactivating mutation in ICP47 is an insertion, deletion, or substitution of a nucleotide in the regulatory sequence of the ICP47 that inactivate the one of the regulatory elements (e.g., a promoter), such that the transcription or translation process of ICP47 is terminated, weakened, or not initiated.

In the present disclosure, the inactivating mutation in ICP47 leads to a reduced level or complete elimination of ICP47 protein, which facilitates antigen presentation and favors immune response to vaccine of the present disclosure. ICP 47 blocks CD8+ T cell recognition of infected cells by inhibiting the transporter associated with antigen presentation. An HSV-1 ICP47 mutant is less neurovirulent than wild-type HSV-1 in mice. The reduced neurovirulence of the ICP47 mutant was due to a protective CD8+ T cell response. When compared with wild-type virus, the ICP47 mutant expressed reduced neurovirulence in immunologically normal mice, and T cell-deficient nude mice after reconstitution with CD8+ T cells. However, the ICP47 mutant exhibited normal neurovirulence in mice that were acutely depleted of CD8+ T cells, and in nude mice that were not reconstituted, or were reconstituted with CD4+ T cells. In contrast, CD8+ T cell depletion did not increase the neurovirulence of an unrelated, attenuated HSV-1 gE mutant. ICP47 was the first viral protein shown to influence neurovirulence by inhibiting CD8+ T cell protection.

In the present disclosure, the inactivating mutation in the other copy of ICP4 in a terminal repeat leads to a reduced level or complete elimination of ICP4 protein. This could be achieved through techniques widely known in the art. For example, in some embodiments, the inactivating mutation is an insertion, deletion, or substitution of a nucleotide in the coding sequence of the ICP4 that causes a missense mutation giving rise to a non-functional ICP4 protein. In some embodiments, the inactivating mutation in ICP4 is an insertion, deletion, or substitution of a nucleotide in the regulatory sequence of the ICP4 that inactivate the one of the regulatory elements (e.g., a promoter), such that the transcription or translation process of ICP4 is terminated, weakened, or not initiated. In some embodiments, the inactivating mutation in ICP4 is a deletion of the coding sequence of ICP 4.

In the present disclosure, a deletion in a coding sequence of ICP4 is made in the genome of the HSV-1 such that other copy of ICP4 is inactivated and the viral vaccine does not express any ICP4 protein. In some embodiments, the deletion in a coding sequence of ICP4 is a deletion of the full-length coding sequence of ICP4. For example, a deletion in a coding sequence of ICP4 is a deletion of nucleotides 146978 to 150886 in the genome of an HSV-1 F strain (GenBank: GU734771.1). In some embodiments, the deletion in a coding sequence of ICP4 is a deletion of a fragment of the coding sequence between nucleotides 146978 and 150886. In some embodiments, the deletion in the coding sequence of ICP4 is a deletion of a fragment of nucleotides overlapping the fragment of nucleotides 146978 and 150886. For example, the deletion in the coding sequence of ICP4 is a deletion of a fragment of nucleotides starting before 146978 but ends between 146978 and 150886. For example, the deletion in the coding sequence of ICP4 is a deletion of a fragment of nucleotides starting between 146978 and 150886 but ends after 150886. In some embodiments, when a deletion of a fragment of nucleotides overlapping the fragment of nucleotides 146978 and 150886 is made, the deletion is made within 146651 to 150948, but leaves the promoter of ICP4 intact and preferably also leaves the polyA sequence and/or TATA box of ICP4 intact. An exemplary promoter sequence of ICP4 is available from GenBank: EF667506.1. It is noted that the specific starting and ending positions of the nucleotides stated in this paragraph is in reference to the F strain, prototype only. When a different strain or isomer is used, the starting and ending positions of the nucleotides of the deletion are to be changed accordingly, which is within ordinary skill of the person in the art.

In some embodiments, the first antigen of the at least one antigen is driven by a promoter of ICP4, and the inactivating mutation in the other copy of ICP4 in the terminal repeat is a deletion in a coding sequence of ICP4 while leaving the promoter of ICP4 intact such that the first antigen of the at least one antigen is operably linked to the promoter of ICP4 in the terminal repeat.

In some embodiments, the first antigen of the at least one antigen is driven by a promoter of an immediate early gene of wild-type HSV-1, such as ICP0, ICP27, ICP4, ICP22 or ICP47, and the first antigen of the at least one antigen together with the driving promoter is inserted into a position corresponds to the deleted internal inverted repeat region.

In some embodiments, the HSV-1 viral vaccine of the present disclosure comprises one copy of ICP34.5, one copy of ICP0, one copy of LAT, and one copy of “a” sequence. In some embodiments, the HSV-1 viral vaccine of the present disclosure comprises UL1 to UL56, and US1 to US11. In some embodiments, the HSV-1 viral vaccine of the present disclosure comprises an engineered gC (UL44) and/or an engineered gE (US8). In some embodiments, the HSV-1 viral vaccine of the present disclosure comprises only one copy of ICP34.5, only one copy of ICP0, one copy of LAT, only one copy of “a” sequence, UL1 to UL56, and US1 to US11. In some embodiments, the HSV-1 viral vaccine of the present disclosure comprises only one copy of ICP34.5, only one copy of ICP0, one copy of LAT, only one copy of “a” sequence, native UL1 to UL43, an engineered UL44, native UL45 to UL56, and native US1 to US11. In some embodiments, the HSV-1 viral vaccine of the present disclosure comprises only one copy of ICP34.5, only one copy of ICP0, one copy of LAT, only one copy of “a” sequence, native UL1 to UL43, an engineered UL44, native UL45 to UL56, and native US1 to US7, an engineered US8, and native US9 to US11.

In some embodiments, the HSV-1 viral vaccine of the present disclosure comprises an engineered UL44. In some embodiments, the HSV-1 viral vaccine of the present disclosure comprises an engineered US8. In some embodiments, the HSV-1 viral vaccine of the present disclosure comprises an engineered UL44 and an engineered US8. In some embodiments, the engineered UL44 has inactivated C3 binding. In some embodiments, the engineered UL44 has a deletion in the C3 binding domain. In some embodiments, the engineered US8 has inactivated FcR binding. In some embodiments, the engineered US8 has a deletion in the FcR binding domain. In some embodiments, the HSV-1 viral vaccine of the present disclosure comprises an engineered UL44 that has a deletion in the C3 binding domain. In some embodiments, the HSV-1 viral vaccine of the present disclosure comprises an engineered US8 that has a deletion in the FcR binding domain. In some embodiments, the HSV-1 viral vaccine of the present disclosure comprises an engineered UL44 that has a deletion in the C3 binding domain, and an engineered US8 that has a deletion in the FcR binding domain.

The HSV-1 viral vaccine of the present disclosure comprises at least one antigen. In some embodiments, the HSV-1 viral vaccine of the present disclosure comprises one antigen (i.e., a first antigen) and the first antigen is driven by the promoter of an immediate early gene, such as the promoter of ICP4. In some embodiments, the first antigen is from a virus, a bacterium or a parasite. In some embodiments, the first antigen is from a non-HSV-1 virus. In some embodiments, the first antigen is from sarbecovirus. In some embodiments, the first antigen is from SARS-Cov, SARS-Cov-2, or variants thereof. In some embodiments, the first antigen is from SARS-Cov, SARS-Cov-2, or variants thereof. In some embodiments, the first antigen is from SARS-Cov-2 or variants thereof. In some embodiments, the first antigen is from SARS-Cov-2 delta or omicron variant. In some embodiments, the first antigen is from SARS-Cov-2 omicron variant. In some embodiments, the first antigen is from SARS-Cov-2 delta variant. In some embodiments, the first antigen is from Spike glycoprotein of sarbecovirus. In some embodiments, the first antigen is from Spike glycoprotein of SARS-Cov, SARS-Cov-2, or variants thereof. In some embodiments, the first antigen is from Spike glycoprotein of SARS-Cov-2 or variants thereof. In some embodiments, the first antigen is from an ectodomain of Spike glycoprotein of sarbecovirus. In some embodiments, the first antigen is from an ectodomain of Spike glycoprotein of SARS-Cov, SARS-Cov-2, or variants thereof. In some embodiments, the first antigen is from an ectodomain of Spike glycoprotein of SARS-Cov-2 or variants thereof. In some embodiments, the first antigen is from NTD domain of Spike glycoprotein of sarbecovirus. In some embodiments, the first antigen is from NTD domain of Spike glycoprotein of SARS-Cov, SARS-Cov-2, or variants thereof. In some embodiments, the first antigen is from NTD domain of Spike glycoprotein of SARS-Cov-2 or variants thereof. In some embodiments, the first antigen is from RBD domain of Spike glycoprotein of sarbecovirus. In some embodiments, the first antigen is from RBD domain of Spike glycoprotein of SARS-Cov, SARS-Cov-2, or variants thereof. In some embodiments, the first antigen is from RBD domain of Spike glycoprotein of SARS-Cov-2 or variants thereof. In some embodiments, the first antigen is from an ectodomain of Spike glycoprotein of delta or omicron variant of SARS-Cov-2. In some embodiments, the first antigen is an ectodomain of Spike glycoprotein of delta variant of SARS-Cov-2.

In some embodiments, the HSV-1 viral vaccine of the present disclosure comprises an ectodomain of Spike glycoprotein of SARS-Cov-2, which is driven by the promoter of ICP4. The ectodomain of Spike glycoprotein of SARS-Cov-2 does not contain the signal sequence, transmembrane domain or the intravirion domain of the Spike glycoprotein. For example, for a delta strain, the ectodomain is from aa 14 to aa 1213 of UniProtKB/Swiss-Prot: PODTC2.1. In some embodiments, the ectodomain of Spike glycoprotein is linked, at N terminus (i.e., the NTD domain), to a signal peptide of gB (UL27) or gD (US6). In some embodiments, the ectodomain of Spike glycoprotein is linked, at C terminus (i.e., the HR2 domain), to a transmembrane-intravirion domain of gB (UL27) or gD (US6). In some embodiments, the ectodomain of Spike glycoprotein is linked, at N terminus (i.e., the NTD domain), to a signal peptide of gB (UL27) or gD (US6), and, at C terminus (i.e., the HR2 domain), to a transmembrane-intravirion domain of gB (UL27) or gD (US6). In some embodiments, the ectodomain of Spike glycoprotein is linked, at N terminus (i.e., the NTD domain), to a signal peptide of gB (UL27), and, at C terminus (i.e., the HR2 domain), to a transmembrane-intravirion domain of gB (UL27). In some embodiments, the ectodomain of Spike glycoprotein is linked, at N terminus (i.e., the NTD domain), to a signal peptide of gD (US6), and, at C terminus (i.e., the HR2 domain), to a transmembrane-intravirion domain of gD (US6).

In some embodiments, the HSV-1 viral vaccine of the present disclosure comprises an ectodomain of Spike glycoprotein of delta variant of SARS-Cov-2, which is driven by the promoter of ICP4. In some embodiments, the HSV-1 viral vaccine of the present disclosure comprises an ectodomain of Spike glycoprotein of delta variant of SARS-Cov-2 with K986P and V987P mutation sites. In some embodiments, the HSV-1 viral vaccine of the present disclosure comprises an ectodomain of Spike glycoprotein of delta variant of SARS-Cov-2 with K986P and V987P mutation sites, as well as 682-GSAS-685 mutations in the furin cleavage site.

In some embodiments, the HSV-1 viral vaccine of the present disclosure comprises an ectodomain of Spike glycoprotein of delta variant of SARS-Cov-2, which is driven by the promoter of ICP4, wherein the ectodomain, with or without the mutations described above, is linked, at N terminus (i.e., the NTD domain), to a signal peptide of gB (UL27). In some embodiments, the HSV-1 viral vaccine of the present disclosure comprises an ectodomain of Spike glycoprotein of delta variant of SARS-Cov-2, which is driven by the promoter of ICP4, wherein the ectodomain, with or without the mutations described above, is linked, at C terminus (i.e., the HR2 domain), to a transmembrane-intravirion domain of gB (UL27). In some embodiments, the HSV-1 viral vaccine of the present disclosure comprises an ectodomain of Spike glycoprotein of delta variant of SARS-Cov-2, which is driven by the promoter of ICP4, wherein the ectodomain, with or without the mutations described above, is linked, at N terminus (i.e., the NTD domain), to a signal peptide of gB (UL27), and, at C terminus (i.e., the HR2 domain), to a transmembrane-intravirion domain of gB (UL27).

In some embodiments, the inverted internal repeat region of the HSV-1 viral vaccine of the present disclosure is replaced by a promoter followed by three repeats of stop codon. In some embodiments, the inverted internal repeat region of the HSV-1 viral vaccine of the present disclosure is replaced by a CMV promoter followed by three repeats of stop codon. In some embodiments, the inverted internal repeat region of the HSV-1 viral vaccine of the present disclosure is replaced by a promoter of an immediate early gene, such as ICP4, followed by the nucleotides encoding the first antigen.

Another aspect of the present disclosure relates to a replication incompetent Herpes Simplex Virus type 1 (HSV-1) viral vaccine, comprising a modified genome of HSV-1 and at least one antigen, wherein the modification comprises a deletion of an internal inverted repeat region which causes deletions of one copy of each of double-copy genes including ICP0, ICP34.5, and ICP4 and latency-associated transcript (LAT), an inactivating mutation in ICP47 and an inactivating mutation in the other copy of ICP4 in a terminal repeat, and wherein a first antigen of the at least one antigen is driven by a promoter of an immediate early gene of wild-type HSV-1, and a second antigen of the at least one antigen is fused into a first HSV-1 glycoprotein.

The terms “deletion of an internal inverted repeat region”, “an inactivating mutation in ICP47”, “an inactivating mutation in the other copy ofICP4”, “a promoter of an immediate early gene”, and “a first antigen” have the same definition and extension as set forth above.

The second antigen is derived from a different source than the first antigen, e.g., a different variant, or a different species. In preferable embodiments, the second antigen is from a virus, a bacterium, or a parasite that belongs to a same family, genus, subgenus, or species of the virus, the bacterium, or the parasite from which the first antigen is derived. For example, in some embodiments, the first and second antigens are from a same genus or subgenus of virus. In some embodiments, the first and second antigens are from sarbecovirus. In some embodiments, the first and second antigens are from SARS-Cov, SARS-Cov-2, or variants thereof. In some embodiments, the first and second antigens are from SARS-Cov, SARS-Cov-2, or variants thereof. In some embodiments, the first and second antigens are from SARS-Cov-2 or variants thereof. In some embodiments, the first and second antigens are from different SARS-Cov-2 variants. In some embodiments, the first and second antigens are from delta and omicron variants of SARS-Cov-2.

In some embodiments, the first antigen is from SARS-Cov-2 (e.g., Wuhan-Hu-1 strain) or variants thereof, and the second antigen is from SARS-Cov or variants thereof. In some embodiments, the first antigen is from SARS-Cov-2, and the second antigen is from SARS-Cov or variants thereof. In some embodiments, the first antigen is from delta or omicron variant of SARS-Cov-2, and the second antigen is from SARS-Cov or variants thereof. In some embodiments, the first antigen is from delta variant of SARS-Cov-2, and the second antigen is from SARS-Cov or variants thereof. In some embodiments, the first antigen is from omicron variant of SARS-Cov-2, and the second antigen is from SARS-Cov or variants thereof. In some embodiments, the first antigen is from spike glycoprotein of SARS-Cov-2 or a variant thereof, and the second antigen is from SARS-Cov or variants thereof. In some embodiments, the first antigen is from spike glycoprotein of SARS-Cov-2, and the second antigen is from SARS-Cov or variants thereof. In some embodiments, the first antigen is from spike glycoprotein of a variant of SARS-Cov-2, and the second antigen is from SARS-Cov or variants thereof. In some embodiments, the first antigen is from spike glycoprotein of delta or omicron variant of SARS-Cov-2, and the second antigen is from SARS-Cov or variants thereof. In some embodiments, the first antigen is an ectodomain of spike glycoprotein of SARS-Cov-2 or a variant thereof, and the second antigen is from SARS-Cov or variants thereof.

In some embodiments, the first antigen is an ectodomain of spike glycoprotein of SARS-Cov-2 or variants thereof, and the second antigen is RBD domain of spike glycoprotein of SARS-Cov or variants thereof. In some embodiments, the first antigen is from an ectodomain of spike glycoprotein of delta or omicron variant of SARS-Cov-2, and the second antigen is from spike glycoprotein of SARS-Cov-2. In some embodiments, the first antigen is an ectodomain of spike glycoprotein of delta or omicron variant of SARS-Cov-2, and the second antigen is NTD domain of spike glycoprotein of SARS-Cov-2. In some embodiments, the first antigen is an ectodomain of spike glycoprotein of delta or omicron variant of SARS-Cov-2, and the second antigen is from spike glycoprotein of SARS-Cov or a variant thereof. In some embodiments, the first antigen is from an ectodomain of spike glycoprotein of delta or omicron variant of SARS-Cov-2, and the second antigen is RBD domain of spike glycoprotein of SARS-Cov. In some embodiments, the first antigen is an ectodomain of spike glycoprotein of omicron variant of SARS-Cov-2, and the second antigen is NTD or RBD domain of spike glycoprotein of delta variant of SARS-Cov-2. In some embodiments, the first antigen is an ectodomain of spike glycoprotein of omicron variant of SARS-Cov-2, and the second antigen is NTD domain of spike glycoprotein of delta variant of SARS-Cov-2.

In some embodiments, the first HSV-1 glycoprotein is gC or gE. In some embodiments, the first HSV-1 glycoprotein is gC or gE and the second antigen is NTD domain of spike glycoprotein of SARS-Cov-2 or a variant thereof or RBD domain of spike glycoprotein of SARS-Cov or a variant thereof. In some embodiments, the first HSV-1 glycoprotein is gE and the second antigen is NTD domain of spike glycoprotein of SARS-Cov-2. In some embodiments, the first HSV-1 glycoprotein is gC and the second antigen is RBD domain of spike glycoprotein of SARS-Cov.

In some embodiments, HSV-1 gC is altered to inactivate the C3 binding. In some embodiments, the inactivation of the C3 binding is achieved by a deletion in the C3 binding domain. In some embodiments, the inactivation of the C3 binding is achieved by a deletion of the binding regions II/III, i.e., from aa 276 to aa 366. In some embodiments, a RBD domain of spike glycoprotein of SARS-Cov (e.g., Tor 2 strain) is inserted in replace of the binding regions II/III.

In some embodiments, HSV-1 gE is altered to inactivate the FcR binding. In some embodiments, the inactivation of the FcR binding is achieved by a deletion in the FcR binding domain. In some embodiments, the inactivation of the FcR binding is achieved by a deletion of aa 235 to 380 in the case of gE of HSV-1 F strain. In some embodiments, an NTD domain of spike glycoprotein of SARS-Cov-2 (e.g., Wuhan-Hu-1 strain) is inserted in replace of the FcR binding domain.

A further aspect of the present disclosure relates to a replication incompetent Herpes Simplex Virus type 1 (HSV-1) viral vaccine, comprising a modified genome of HSV-1 and at least one antigen, wherein the modification comprises a deletion of an internal inverted repeat region which causes deletions of one copy of each of double-copy genes including ICP0, ICP34.5, and ICP4 and latency-associated transcript (LAT), an inactivating mutation in ICP47 and an inactivating mutation in the other copy of ICP4 in a terminal repeat, and wherein a first antigen of the at least one antigen is driven by a promoter of an immediate early gene of wild-type HSV-1, a second antigen of the at least one antigen is fused into a first HSV-1 glycoprotein, and a third antigen of the at least one antigen is fused into a second HSV-1 glycoprotein.

The terms “deletion of an internal inverted repeat region”, “an inactivating mutation in ICP47”, “an inactivating mutation in the other copy of ICP4”, “a promoter of an immediate early gene”, “a first antigen”, and “a second antigen” have the same definition and extension as set forth above.

The first, second, and third antigens are derived from different sources from each other, e.g., different variants, or different species. In preferable embodiments, the first, second, and third antigens are from a virus, a bacterium, or a parasite that belongs to a same family, genus, subgenus, or species. For example, in some embodiments, the first, second, and third antigens are from a same genus or subgenus of virus. In some embodiments, the first, second, and third antigens are from sarbecovirus. In some embodiments, the first, second, and third antigens are from SARS-Cov, SARS-Cov-2, or variants thereof. In some embodiments, the first, second, and third antigens are from SARS-Cov-2 or variants thereof. In some embodiments, the first, second, and third antigens are from different SARS-Cov-2 variants. In some embodiments, at least two of the first, second, and third antigens are from delta and omicron variants of SARS-Cov-2. In some embodiments, two of the first, second, and third antigens are from delta and omicron variants of SARS-Cov-2, and other is from SARS-Cov or a variant thereof.

In some embodiments, the first antigen is an ectodomain of spike glycoprotein of SARS-Cov-2 or a variant thereof, the second antigen is a NTD domain of spike glycoprotein of SARS-Cov-2 or a variant thereof which is different from the SARS-Cov-2 or a variant thereof from which the first antigen is derived, and the third antigen is a RBD domain of spike glycoprotein of SARS-Cov or a variant thereof.

In some embodiments, the first antigen is an ectodomain of spike glycoprotein of delta variant of SARS-Cov-2, the second antigen is a NTD domain of spike glycoprotein of SARS-Cov-2, and the third antigen is a RBD domain of spike glycoprotein of SARS-Cov or a variant thereof. In some embodiments, the first antigen is an ectodomain of spike glycoprotein of delta variant of SARS-Cov-2, the second antigen is a RBD domain of spike glycoprotein of SARS-Cov or a variant thereof, and the third antigen is a NTD domain of spike glycoprotein of SARS-Cov-2.

In some embodiments, the first and second HSV-1 glycoproteins are gC and gE. In some embodiments, the first HSV-1 glycoprotein is gC and the second HSV-1 glycoprotein is gE. In some embodiments, the first HSV-1 glycoprotein is gE and the second HSV-1 glycoprotein is gC.

In some embodiments, the first HSV-1 glycoprotein is gE, the second antigen is an NTD domain of spike glycoprotein of SARS-Cov-2 or a variant thereof, the second HSV-1 glycoprotein is gC, and the third antigen is a RBD domain of spike glycoprotein of SARS-Cov or a variant thereof.

In some embodiments, HSV-1 gC is altered to inactivate the C3 binding. In some embodiments, the inactivation of the C3 binding is achieved by a deletion in the C3 binding domain. In some embodiments, the inactivation of the C3 binding is achieved by a deletion the binding regions II/III, i.e., from aa 276 to aa 366. In some embodiments, a RBD domain of spike glycoprotein of SARS-Cov (e.g., Tor 2 strain) is inserted in replace of the binding regions II/III.

In some embodiments, HSV-1 gE is altered to inactivate the FcR binding. In some embodiments, the inactivation of the FcR binding is achieved by a deletion in the FcR binding domain. In some embodiments, the inactivation of the FcR binding is achieved by a deletion of aa 235 to 380 in the case of gE of HSV-1 F strain. In some embodiments, an NTD domain of spike glycoprotein of SARS-Cov-2 (e.g., Wuhan-Hu-1 strain) is inserted in replace of the FcR binding domain.

In some embodiments, the first HSV-1 glycoprotein is gE, the second antigen is an NTD domain of spike glycoprotein of SARS-Cov-2 (e.g., Wuhan-Hu-1 strain) that is inserted in replace of the FcR binding domain, the second HSV-1 glycoprotein is gC, and the second antigen is a RBD domain of spike glycoprotein of SARS-Cov (e.g., Tor 2 strain) that is inserted in replace of the binding regions II/III of the C3 binding domain.

### Vaccine Composition

A further aspect of the disclosure is related to a vaccine composition which comprises the HSV-1 viral vaccine as described herein and a pharmaceutically acceptable carrier. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing. Formulated compositions, especially liquid formulations, may contain a bacteriostat to prevent or minimize degradation during storage, including but not limited to effective concentrations (usually 1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The vaccine compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. The vaccine composition may optionally include an adjuvant to enhance an immune response of the host. Suitable adjuvants are, for example, toll-like receptor (TLR) agonists, alum, AlPO4, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, Ind.) and IL-12 (Genetics Institute, Cambridge, Mass.), among many other suitable adjuvants well known in the art, may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product. In some embodiments, the immunogenic compositions of the disclosure may include or be administered with more than one adjuvant. In some embodiments, the immunogenic compositions of the disclosure may include or be administered with two adjuvants. In some embodiments, the immunogenic compositions of the disclosure may include or be administered with a plurality of adjuvants.

For vaccine compositions, examples of suitable adjuvants include, e.g., aluminum hydroxide, lecithin, Freund's adjuvant, MPL™ and IL-12. In some embodiments, the vaccine compositions disclosed herein (e.g., SARS-Cov-2 vaccine composition) can be formulated as a controlled-release or time-release formulation. This can be achieved in a composition that contains a slow-release polymer or via a microencapsulated delivery system or bio-adhesive gel. The various vaccine compositions can be prepared in accordance with standard procedures well known in the art.

In some embodiments, the vaccine compositions of the disclosure can contain an adjuvant formulation comprising a metabolizable oil (e.g., squalene) and alpha tocopherol in the form of an oil-in-water emulsion, and polyoxyethylene sorbitan monooleate (Tween-80). In some embodiments, the adjuvant formulation can comprise from about 2% to about 10% squalene, from about 2 to about 10% alpha tocopherol (e.g., D-alpha-tocopherol) and from about 0.3 to about 3% polyoxyethylene sorbitan monooleate. In some embodiments, the adjuvant formulation can comprise about 5% squalene, about 5% tocopherol, and about 0.4% polyoxyethylene sorbitan monooleate. In some embodiments, the immunogenic compositions of the disclosure can contain 3 de-O-acylated monophosphoryl lipid A (3D-MPL), and an adjuvant in the form of an oil in water emulsion, which adjuvant contains a metabolizable oil, alpha tocopherol, and polyoxyethylene sorbitan monoleate. In some embodiments, the vaccine compositions of the disclosure can contain QS21 (extract of Quillaja saponaria Molina: fraction 21), 3D-MPL and an oil in water emulsion wherein the oil in water emulsion comprises a metabolizable oil, alpha tocopherol and polyoxyethelene sorbitan monooleate. In some embodiments, the vaccine compositions of the disclosure can contain QS21, 3D-MPL and an oil in water emulsion wherein the oil in water emulsion has the following composition: a metabolisible oil, such as squalene, alpha tocopherol and Tween-80. In some embodiments, the vaccine compositions of the disclosure can contain an adjuvant in the form of a liposome composition.

In some embodiments, the vaccine compositions of the disclosure can contain an adjuvant formulation comprising a metabolizable oil (e.g., squalene), polyoxyethylene sorbitan monooleate (Tween-80), and Span 85. In some embodiments, the adjuvant formulation can comprise about 5% (w/v) squalene, about 0.5% (w/v) polyoxyethylene sorbitan monooleate, and about 0.5% (w/v) Span 85.

In some embodiments, the vaccine compositions of the disclosure can contain an adjuvant formulation comprising Quillaja saponins, cholesterol, and phosphorlipid, e.g., in the form of a nanoparticle composition. In some embodiments, vaccine compositions of the disclosure can contain a mixture of separately purified fractions of Quillaja saponaria Molina where are subsequently formulated with cholesterol and phospholipid. In some embodiments, the vaccine compositions of the disclosure can contain an adjuvant selected from the group consisting of MF59™, Matrix-A™, Matrix-C™, Matrix-M™, AS01, AS02, AS03, and AS04.

One or more adjuvants may be used in combination and may include, but are not limited to, alum (aluminum salts), oil-in-water emulsions, water-in-oil emulsions, liposomes, and microparticles, such as poly(lactide-co-glycolide) microparticles. In some embodiments, the vaccine compositions further comprise an aluminum salt adjuvant. In some embodiments, the aluminum salt adjuvant comprises one or more of the group consisting of amorphous aluminum hydroxyphosphate sulfate, aluminum hydroxide, aluminum phosphate, and potassium aluminum sulfate. In some embodiments, the aluminum salt adjuvant comprises one or both of aluminum hydroxide and aluminum phosphate. In some embodiments, the aluminum salt adjuvant comprises aluminum hydroxide. In some embodiments, a unit dose of the vaccine composition comprises from about 0.25 to about 0.50 mg Al³⁺, or about 0.35 mg Al³⁺. In some embodiments, the vaccine composition further comprises an additional adjuvant.

### Methods and Uses

In some embodiments, provided herein is a method for generating an immune response in a subject, comprising administering to the subject an effective amount of an HSV-1 viral vaccine or the vaccine composition described herein.

In some embodiments, provided herein is a method for generating an immune response to a surface antigen of a coronavirus in a subject, wherein the surface antigen comprises an S protein or antigenic fragment thereof, and the method comprises administering to the subject an effective amount of an HSV-1 viral vaccine or the vaccine composition described herein.

A subject can be selected for treatment that has, or is at risk for developing infection with the coronavirus, for example because of exposure or the possibility of exposure to the coronavirus. Following administration of a disclosed vaccine, the subject can be monitored for infection or symptoms associated with coronavirus, or both.

Typical subjects intended for treatment with the vaccines and methods of the present disclosure include humans, as well as non-human primates and other animals. To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize coronavirus infection. These and other routine methods allow the clinician to select patients in need of therapy using the methods and pharmaceutical compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The administration of a disclosed vaccine can be for prophylactic or therapeutic purpose. When provided prophylactically, the disclosed vaccines are provided in advance of any symptom, for example, in advance of infection. The prophylactic administration of the disclosed vaccine serves to prevent or ameliorate any subsequent infection. When provided therapeutically, the disclosed vaccines are provided at or after the onset of a symptom of disease or infection, for example, after development of a symptom of infection with coronavirus, or after diagnosis with the coronavirus infection. The vaccines can thus be provided prior to the anticipated exposure to coronavirus so as to attenuate the anticipated severity, duration or extent of an infection and/or associated disease symptoms, after exposure or suspected exposure to the virus, or after the actual initiation of an infection.

The vaccines described herein are provided to a subject in an amount effective to induce or enhance an immune response against, e.g., the coronavirus in the subject, preferably a human. The actual dosage of vaccine will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

There can be several boosts. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example, a relatively large dose in a primary immunization and then a boost with relatively smaller doses.

In some embodiments, the boost can be administered about two, about three to eight, or about four, weeks following the prime, or about several months after the prime. In some embodiments, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., prevention of infection or improvement in disease state (e.g., reduction in viral load). If such monitoring indicates that vaccination is sub-optimal, the subject can be boosted with an additional dose of vaccine, and the vaccination parameters can be modified in a fashion expected to potentiate the immune response.

The amount utilized in a vaccine composition is selected based on the subject population (e.g., infant or elderly). An optimal amount for a particular composition can be ascertained by standard studies involving observation of antibody titers and other responses in subjects. It is understood that a therapeutically effective amount of a disclosed vaccine in a vaccine composition can include an amount that is ineffective at eliciting an immune response by administration of a single dose, but that is effective upon administration of multiple dosages, for example in a prime-boost administration protocol.

Upon administration of a disclosed vaccine of this disclosure, the immune system of the subject typically responds to the vaccine by producing antibodies specific for e.g., the coronavirus S protein peptide included in the vaccine. Such a response signifies that an immunologically effective dose was delivered to the subject. In some embodiments, the antibody response of a subject will be determined in the context of evaluating effective dosages/immunization protocols. In most instances it will be sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum which bind to an antigen.

In some embodiments, the coronavirus infection does not need to be completely eliminated or reduced or prevented for the methods to be effective. For example, elicitation of an immune response to a coronavirus with one or more of the disclosed vaccines can reduce or inhibit infection with the coronavirus by a desired amount, for example, by at least 10%, at least 20%, at least 50%, at least 60%, at least 70%, at least 80%, at least 90%, at least 95%, at least 98%, or even at least 100% (elimination or prevention of detectable infected cells), as compared to infection with the coronavirus in the absence of the immunogen. In additional examples, coronavirus replication can be reduced or inhibited by the disclosed methods. Coronavirus replication does not need to be completely eliminated for the method to be effective. For example, the immune response elicited using one or more of the disclosed immunogens can reduce replication of the corresponding coronavirus by a desired amount, for example, by at least 10%, at least 20%, at least 50%, at least 60%, at least 70%, at least 80%, at least 90%, at least 95%, at least 98%, or even at least 100% (elimination or prevention of detectable replication of the coronavirus), as compared to replication of the coronavirus in the absence of the immune response.

In some embodiments, administration of a therapeutically effective amount of one or more of the disclosed vaccines to a subject induces a neutralizing immune response in the subject. To assess neutralization activity, following immunization of a subject, serum can be collected from the subject at appropriate time points, frozen, and stored for neutralization testing. Methods to assay for neutralization activity are known to the person of ordinary skill in the art and are further described herein, and include, but are not limited to, plaque reduction neutralization (PRNT) assays, microneutralization assays, flow cytometry-based assays, single-cycle infection assays. In some embodiments, the serum neutralization activity can be assayed using a panel of coronavirus pseudo-viruses.

In some embodiments, a neutralizing immune response induced by the disclosed vaccines herein generates a neutralizing antibody against a coronavirus such as SARS-Cov-2. In some embodiments, the neutralizing antibody herein binds to a cellular receptor or coreceptor of a coronavirus such as SARS-Cov-2 or component thereof. In some embodiments, the viral receptor or coreceptor is a coronavirus receptor or coreceptor, preferably a pneumonia virus receptor or coreceptor, more preferably a human coronavirus receptor such as SARS-Cov-2 receptor or coreceptor. In some embodiments, the neutralizing antibody herein modulates, decreases, antagonizes, mitigates, blocks, inhibits, abrogates and/or interferes with at least one coronavirus such as SARS-Cov-2 activity or binding, or with a coronavirus such as SARS-Cov-2 receptor activity or binding, in vitro, in situ and/or in vivo, such as SARS-Cov-2 release, SARS-Cov-2 receptor signaling, membrane SARS-Cov-2 cleavage, SARS-Cov-2 activity, SARS-Cov-2 production and/or synthesis. In some embodiments, the disclosed vaccines herein induce neutralizing antibodies against SARS-Cov-2 that modulate, decrease, antagonize, mitigate, block, inhibit, abrogate and/or interfere with SARS-Cov-2 binding to a SARS-Cov-2 receptor or coreceptor, such as angiotensin converting enzyme 2 (ACE2), dipeptidyl peptidase 4 (DPP4), dendritic cell-specific intercellular adhesion molecule-3-grabbing non integrin (DC-SIGN), and/or liver/lymph node-SIGN (L-SIGN).

In some embodiments, a neutralizing immune response induced by the disclosed vaccines herein generates a neutralizing antibody against sarbecovirus including SARS-Cov, SARS-Cov-2 and variants thereof. In this sense, the vaccines provided herein is a pan-sarbecovirus vaccine.

Such vaccine compositions can be administered to subjects by a variety of administration modes known to the person of ordinary skill in the art, for example, intramuscular, intradermal, subcutaneous, intravenous, intra-arterial, intra-articular, intraperitoneal, intranasal, sublingual, tonsillar, oropharyngeal, or other parenteral and mucosal routes.

### Sequences

gD-S^{ECTO} amino acid sequence (SEQ ID NO: 1)
MGGAAARLGAVILFVVIVGLHGVRGQCVNLRTRTQLPPAYTNSFTRGVYYPDKVERS
SVLHSTQDLFLPFFSNVTWFHAIHVSGTNGTKRFDNPVLPENDGVYFASTEKSNIIR
GWIFGTTLDSKTQSLLIVNNATNVVIKVCEFQFCNDPFLDVYYHKNNKSWMESGVYS
SANNCTFEYVSQPFLMDLEGKQGNFKNLREFVFKNIDGYFKIYSKHTPINLVRDLPQ
GFSALEPLVDLPIGINITRFQTLLALHRSYLTPGDSSSGWTAGAAAYYVGYLQPRTF
LLKYNENGTITDAVDCALDPLSETKCTLKSFTVEKGIYQTSNERVQPTESIVRFPNI
TNLCPFGEVENATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVG
GNYNYRYRLFRKSNLKPFERDISTEIYQAGSKPCNGVEGENCYFPLQSYGFQPTNGV
GYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQGVNC
TEVPVAIHADQLTPTWRVYSTGSNVFQTRAGCLIGAEHVNNSYECDIPIGAGICASY
QTQTNSRGSASSVASQSIIAYTMSLGAENSVAYSNNSIAIPTNFTISVTTEILPVSM
TKTSVDCTMYICGDSTECSNLLLQYGSFCTQLNRALTGIAVEQDKNTQEVFAQVKQI
YKTPPIKDFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQNVVNQNAQAL
NTLVKQLSSNFGAISSVLNDILSRLDPPEAEVQIDRLITGRLQSLQTYVTQQLIRAA
EIRASANLAATKMSECVLGQSKRVDFCGKGYHLMSFPQSAPHGVVFLHVTYVPAQEK
NFTTAPAICHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNIQKEIDRLN

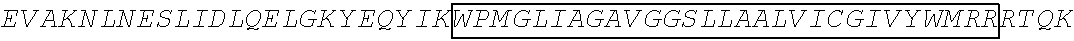

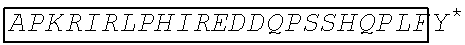

Note: gD signal peptide is underlined; ectodomain of
SARS-Cov-2 delta variant is italic; gD transmembrane-
intravirion domain is boxed; ^{*}represents stop codon.
gB-S^{ECTO} amino acid sequence (SEQ ID NO: 2)
MRQGAPARGCRWFVVWALLGLTLGVLVASAAQCVNLRTRTQLPPAYTNSFTRGVYYP
DKVFRSSVLHSTQDLFLPFFSNVTWFHAIHVSGTNGTKRFDNPVLPENDGVYFASTE
KSNIIRGWIFGTTLDSKTQSLLIVNNATNVVIKVCEFQFCNDPFLDVYYHKNNKSWM
ESGVYSSANNCTFEYVSQPFLMDLEGKQGNFKNLREFVFKNIDGYFKIYSKHTPINL
VRDLPQGFSALEPLVDLPIGINITRFQTLLALHRSYLTPGDSSSGWTAGAAAYYVGY
LQPRTFLLKYNENGTITDAVDCALDPLSETKCTLKSFTVEKGIYQTSNERVQPTESI
VRFPNITNLCPFGEVENATRFASVYAWNRKRISNCVADYSVLYNSASFSTFKCYGVS
PTKLNDLCFTNVYADSFVIRGDEVRQIAPGQTGKIADYNYKLPDDFTGCVIAWNSNN
LDSKVGGNYNYRYRLFRKSNLKPFERDISTEIYQAGSKPCNGVEGENCYFPLQSYGF
QPTNGVGYQPYRVVVLSFELLHAPATVCGPKKSTNLVKNKCVNFNFNGLTGTGVLTE
SNKKFLPFQQFGRDIADTTDAVRDPQTLEILDITPCSFGGVSVITPGTNTSNQVAVL
YQGVNCTEVPVAIHADQLTPTWRVYSTGSNVFQTRAGCLIGAEHVNNSYECDIPIGA
GICASYQTQTNSRGSASSVASQSIIAYTMSLGAENSVAYSNNSIAIPTNFTISVTTE
ILPVSMTKTSVDCTMYICGDSTECSNLLLQYGSFCTQLNRALTGIAVEQDKNTQEVF
AQVKQIYKTPPIKDFGGFNFSQILPDPSKPSKRSFIEDLLENKVTLADAGFIKQYGD
CLGDIAARDLICAQKFNGLTVLPPLLTDEMIAQYTSALLAGTITSGWTFGAGAALQI
PFAMQMAYRENGIGVTQNVLYENQKLIANQFNSAIGKIQDSLSSTASALGKLQNVVN
QNAQALNTLVKQLSSNFGAISSVLNDILSRLDPPEAEVQIDRLITGRLQSLQTYVTQ
QLIRAAEIRASANLAATKMSECVLGQSKRVDFCGKGYHLMSFPQSAPHGVVFLHVTY
VPAQEKNFTTAPAICHDGKAHFPREGVFVSNGTHWFVTQRNFYEPQIITTDNTFVSG
NCDVVIGIVNNTVYDPLQPELDSFKEELDKYFKNHTSPDVDLGDISGINASVVNIQK

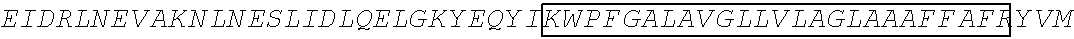

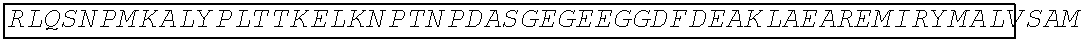

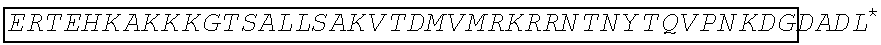

Note: gB signal peptide is underlined; ectodomain of
SARS-Cov-2 delta variant is italic;
gB transmembrane-
intravirion domain is boxed; ^{*}represents stop codon.
gC-S^{RBD} amino acid sequence (SEQ ID NO: 3)
MAPGRVGLAVVLWSLLWLGAGVSGGSETASTGPTITAGAVINASEAPTSGSPGSAAS
PEVTPTSTPNPNNVTQNKTTPTEPASPPTTPKPTSTPKSPPTSTPDPKPKNNTTPAK
SGRPTKPPGPVWCDRRDPLARYGSRVQIRCRERNSTRMEFRLQIWRYSMGPSPPIAP
APDLEEVLTNITAPPGGLLVYDSAPNLTDPHVLWAEGAGPGADPPLYSVTGPLPTQR
LIIGEVTPATQGMYYLAWGRMDSPHEYGTWVRVRMFRPPSLTLQPHRVVPSGDVVRF
PNITNLCPFGEVENATKFPSVYAWERKKISNCVADYSVLYNSTFFSTEKCYGVSATK
LNDLCFSNVYADSFVVKGDDVRQIAPGQTGVIADYNYKLPDDFMGCVLAWNTRNIDA
TSTGNYNYKYRYLRHGKLRPFERDISNVPFSPDGKPCTPPALNCYWPLNDYGFYTTT
GIGYQPYRVVVLSFELLNAPATVCGPKLSTDLIKNQCVNFLVLPRPTITMEFGVRHV
VCTAGCVPEGVTFAWFLGDDPSPAAKSAVTAQESCDHPGLATVRSTLPISYDYSEYI
CRLTGYPAGIPVLEHHGSHQPPPRDPTERQVIEAIEWVGIGIGVLAAGVLVVTAIVY
VVRTSQSRQRHRR^{*}
Note: SARS-Cov Tor2 RBD domain is italic; ^{*}represents
stop codon.
gE-S^{NTD} amino acid sequence (SEQ ID NO: 4)
MDRGAVVGFLLGVCVVSCLAGTPKTSWRRVSVGEDVSLLPAPGPTGRGPTQKLIWAV
EPLDGCGPLHPSWXSLMPPKQVPETVVDAACMRAPVPLAMAYAPPAPSATGGLRIDF
VWQERAAVVNRSLVIYGVRETDSGLYTLSVGDIKDPARQVASVVLVVQPAPVPTPPP
TPADYDEDDNDEGEGEDESLAGTPASGTPRLPPPPAPPRSWPSAPEVSHVRGVTVRM
ETPEAILFQCVNLITRTQLPPAYINSFTRGVYYPDKVERSSVLASTQDLFLPFFSNV
TWFHAIHVSGINGTKRFDNPVLPENDGVYFASTEKSNIIRGWIFGTTLDSKTQSLLI
VNNATNVVIKVCEFQFCNDPFLGVYYHKNNKSWMESEFRVYSSANNCTFEYVSQPFL
MDLEGKQGNFKNLREFVEKNIDGYFKIYSKHTPINLVRDLPQGFSALEPLVDLPIGI
NITRFQTLLALHRSYLIPGDSSSGWTAGAAAYYVGYLQPRTELLKYNENGTITDAVD
CALDPLSETKCTLKSYRNAVVEQPLPQRGADLAEPTHPHVGAPPHAPPTHGALRLGA
VMGAALLLSVLGLSVWACMTCWRRRAWRAVKSRASGKGPTYIRVADSELYADWSSDS
EGERDQVPWLAPPERPDSPSTNGSGFEILSPTAPSVYPRSDGHQSRRQLTTFGSGRP
DRRYSQASDSSVFW^{*}
Note: SARS-Cov-2 Wuhan-Hu-1 NTD domain is italic;
^{*}represents stop codon.

### EXAMPLES

### Constructions of Non-Replicating HSV-1 Viral Vector MVR-ΔIR4 and MVR-ΔIR47.

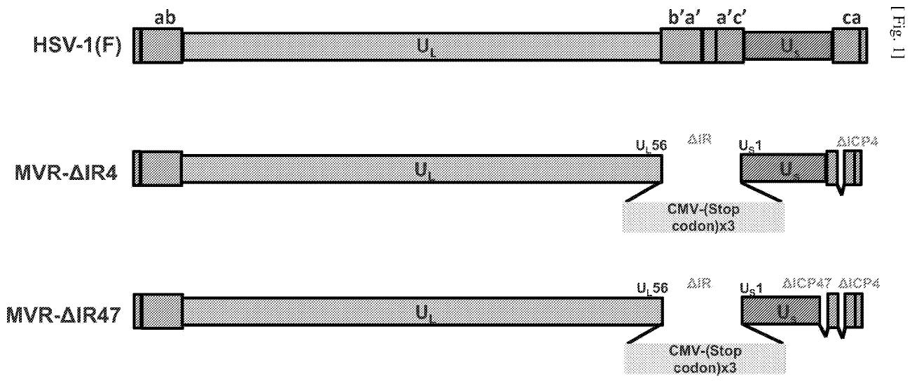

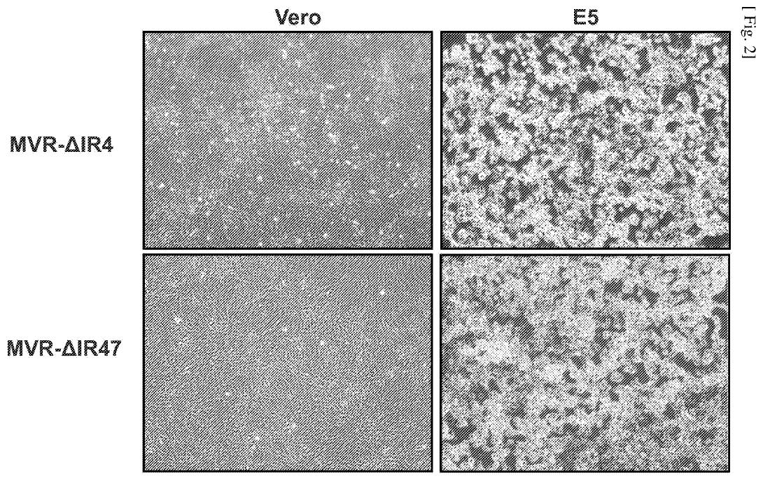

FIG. 1 shows schematic diagrams of the HSV-1 (F), MVR-ΔIR4 and MVR-ΔIR47 genomes. The non-replicating HSV-1 viral vector MVR-ΔIR4 comprises the deletion of ICP4 gene in terminal repeat (TR) region, and a modified internal repeat (IR) region which containing one copy gene encoding ICP0, ICP4, ICP34.5, LAT, ORF P and ORF O replaced by the CMV promoter followed by three repeat stop codon. The non-replicating HSV-1 viral vector MVR-ΔIR47 comprises the additional deletion of ICP47 gene based on the vector of MVR-ΔIR4. The recombinant virus was constructed in several steps with the aid of bacterial artificial chromosome (BAC) system.

The details of viral construction are described following. A HSV-1 (F) in prototype (P) arrangement is used. CMV cassettes flanked by upstream of nucleotides 117005 and downstream of nucleotides 132096 in the context of a wild type genome were PCR amplified from HSV-1 viral genome by two sets of primers respectively (GAAGATCTAATATTTTTATTGCAACTCCCTG (SEQ ID NO: 5), CTAGCTAGCTTATAAAAGGCGCGTCCCGTGG (SEQ ID NO: 6)) and (GCTCTAGATTGCGACGCCCCGGCTC (SEQ ID NO: 7), CCTTAATTAAGGTTACCACCCTGTAGCCCCGATGT (SEQ ID NO: 8)) and inserted into a gene replacement plasmid pKO5 to generate pKO-CMV-STOP. pKO-CMV-STOP was then transfected to Escherichia coli with BAC-HSV-1 (F) by electroporation to generate BAC-CMV-STOP. Then, the pKO-AICP4 plasmid which containing 5′-Flanking sequence of nucleotides 145867-146977 and 3′-Flanking sequence of nucleotides 150887-151868 in the context of a wild type genome were PCR amplified from HSV-1 viral genome by two sets of primers respectively (ATCCCGAGCCGGGGCGTCGCGATGCCGA (SEQ ID NO: 9), CGCCGATGCGGGGCGATCCTCCGGGGATACGGCTGC (SEQ ID NO: 10)) and (CGGGCCGGGACGGGGCGGGGCGCTTGCGAAAC (SEQ ID NO: 11), AACGCCCGCCGCGCGCGCGCACGCCGCCCGGACC (SEQ ID NO: 12)) transfected to Escherichia coli harboring BAC-CMV-STOP by electroporation to generate BAC-ΔIR4. MVR-ΔIR4 virus was obtained by transfection of BAC-ΔIR4 plasmid following by amplification in E5 cells (Vero-derived, ICP4 complementing cell line).

MVR-ΔIR47 was additional deletion of ICP47 gene based on the vector of MVR-ΔIR4. The pKO-ΔICP47 plasmid which containing 5′-Flanking sequence of nucleotides 146977-145867 and 3′-Flanking sequence of nucleotides 145088-143761 in the context of a wild type genome were PCR amplified from HSV-1 viral genome by two sets of primers respectively (CGCCGATGCGGGGCGATCCTCCGGGGATACGG (SEQ ID NO: 13), TCCCGAGCCGGGGCGTCGCGATGCCGACGCCG (SEQ ID NO: 14)) and (ATGAGCCAGACCCAACCCCCGGCCCCAGTTGG (SEQ ID NO: 15), CAGAAAATGTAACCATACCCAAACCGACTCT (SEQ ID NO: 16)) transfected to Escherichia coli harboring BAC-ΔIR4 by electroporation to generate BAC-ΔIR47. MVR-ΔIR47 virus was obtained by transfection of BAC-ΔIR47 plasmid following by amplification in E5 cells (Vero-derived, ICP4 complementing cell line).

### Constructions of Non-Replicating HSV-1 Based Sarbecovirus Spike Protein Vaccine Virus MVR-S-gB and MVR-S-gD.

The MVR-S-gB is a MVR-A IR47 based virus which consists of the ectodomain of SARS-Cov-2 Delta strain B.1.617.2 spike and the transmembrane domain (TM) and cytoplasmic tail (CT) of the gB protein. The MVR-S-gD is a MVR-ΔIR47 based virus which consists of the ectodomain of SARS-Cov-2 Delta strain B.1.617.2 spike and the transmembrane domain (TM) and cytoplasmic tail (CT) of the gD protein.

The pKO-gC-S^{RBD} plasmid in which the S^{RBD} cassette was flanked by 5′-Flanking sequence of nucleotides 95401-97031 and 3′-Flanking sequence of nucleotides 97311-98692 in the context of a wild type genome were PCR amplified from HSV-1 viral genome by two sets of primers respectively (GGGCCACCGTCCCCCCCGACACCCCAACGA (SEQ ID NO: 17), GTGGGGCTGGAGGGTCAGAGACGGGGGGCGG (SEQ ID NO: 18)) and (CTGGTGCTGCCGCGGCCAACCATCACCATG (SEQ ID NO: 19), ACGCCTCCACCCGTGCTGCCGTCGCTAGAC (SEQ ID NO: 20)) transfected to Escherichia coli harboring BAC-ΔIR47 by electroporation to generate BAC-ΔIR47-SgC.

The pKO-gE-S^{NTD} plasmid in which the S^{NTD} cassette was flanked by 5′-Flanking sequence of nucleotides 140341-141812 and 3′-Flanking sequence of nucleotides 142251-143580 in the context of a wild type genome were PCR amplified from HSV-1 viral genome by two sets of primers respectively (AAGCATCGACCACACCCTTCCCCACGGGA (SEQ ID NO: 21), AAACAGGATAGCTTCCGGAGTCTCCATACGCA (SEQ ID NO: 22)) and (TACCGGAACGCGGTGGTGGAACAGCCCCTC (SEQ ID NO: 23), AAAAATCAACCGGGAGACAACATTGCCAAT (SEQ ID NO: 24)) transfected to Escherichia coli harboring BAC-ΔIR47-SgC by electroporation to generate BAC-A IR47-SgC-SgE.

The pKO-gB-S^{ECTO} or pKO-gD-S^{ECTO} plasmid in which the S^{ECTO} cassette was flanked by 5′-Flanking sequence of nucleotides 145867-146977 and 3′-Flanking sequence of nucleotides 150887-151868 in the context of a wild type genome were PCR amplified from HSV-1 viral genome by two sets of primers respectively (ATCCCGAGCCGGGGCGTCGCGATGCCGA (SEQ ID NO: 25), CGCCGATGCGGGGCGATCCTCCGGGGATACGGCTGC (SEQ ID NO: 26)) and (CGGGCCGGGACGGGGCGGGGCGCTTGCGAAAC (SEQ ID NO: 27), AACGCCCGCCGCGCGCGCGCACGCCGCCCGGACC (SEQ ID NO: 28)) transfected to Escherichia coli harboring BAC-ΔIR47-SgC-SgE by electroporation to generate BAC-S-gB or BAC-S-gD. MVR-S-gB and MVR-S-gD virus was obtained by transfection of BAC-S-gB or BAC-S-gD plasmid following by amplification in E5 cells (Vero-derived, ICP4 complementing cell line).

### Materials and Methods

### Microscopy Analysis

The morphological phenotypes of infected cells were documented by EVOS XL core Imaging system. For analyses of infected cells phenotypes, Vero or E5 cells were grown in 150 cm² flask and infected with MVR-ΔIR4 or MVR-ΔIR47 viruses. At 48 hours post infection, the morphology of each cell was observed by EVOS XL core microscopy and raw digital images were documented.

### Immunoblotting Assay

75 cm² flasks were seeded with 6×10⁶ of E5 or Vero cells. After overnight incubation at 37° C., cells were mock infected or infected at 10 PFU of HSV-1 (F), MVR-S-gB, MVR-S-gD per cell. The cells were harvested at 24 hours post infection and lysed with the RIPA buffer (Beyotime). The cell lysates were heated at 100° C. for 10 minutes and then loaded on 8% SDS-PAGE gel. The proteins were transferred onto polyvinylidene difluoride (PVDF) membrane (Minipore), and the membrane was blocked with 5% milk in PBST for 1 hour at room temperature (RT), and reacted with primary antibodies against SARS-Cov-2 Spike Protein (Cat. #42172, CST), gE (Cat. #ab6510, Abcam), gC (Cat. #ab6509, Abcam), ICP4 (Cat. #ab6514, Abcam), ICP27 (Cat. #ab53480, Abcam), ICP8 (Cat. #ab20194, Abcam), ICP0 (Cat. #ab6513, Abcam), gD (Cat. #sc-21719, Santa Cruz), or GAPDH (Cat. #2118S, CST) at 4° C. overnight. The membrane was then washed with PBST on a shaker 3 times and incubated with HRP-conjugated goat anti-Mouse IgG (Cat. #31430, Invitrogen) or HRP-conjugated goat anti-Rabbit IgG (Cat. #31460, Invitrogen) for 1 hour at RT. The membrane was then washed with PBST on a shaker 3 times and added ECL Western Blotting Substrate (Cat. #WBKLS0500, Minipore), the blots were imaged by ChemiDoc XRS+ (Bio-rad).

Mouse immunization study. Female BalB/c mice were used in the study. For intramuscular vaccination using the MVR-S-gB or negative control d120 virus was prepared in 200 μL total volume with or without aluminum as the adjuvant. Three immunizations were performed for all the mice at a 14-day interval. Blood was collected from the orbital sinus on study days 0, 8, 14, 22, 28, 36, 43 and 50. Sera were isolated by low-speed centrifugation and stored at −80° C. until use.

Pseudotyped virus neutralization assays. The PNA was performed by Genscript using a lentivirus, which encoding luciferase and displaying the spike protein of Wuhan-Hu-1 strain, Delta strain or Omicron BA1 strain respectively. To measure neutralizing antibody activity in serum, mouse sera were diluted at 1:50 in opti-MEM buffer. Thereafter, a 25 μL aliquot of diluted sera, positive control ACE2 protein or negative control assay buffer was incubated with a 25 μL aliquot of HIV-based SARS-Cov-2 pseudovirus containing 500 TCID50 for 1 h at RT in a 96-well plate. At the end of the incubation, 50 μL of the mixture was added to Opti-HEK293/ACE2 target cells and incubated for 24 hours at 37° C. Thereafter, cells were analyzed by luciferase assays. The data was analyzed using GraphPad Prism 6.0. The luminescence intensity of the negative group was used as the cutoff value to identify the positivity of the neutralization antibodies.

### The Non-Replicating HSV-1 Viral Vector Only Replicates in Viral ICP4 Complementing Cells

Vero or E5 cells were seeded into 150 cm² flask at a density of 1.2×10⁷ cells/cm². After overnight incubation, the cells were infected at 1 PFU of MVR-ΔIR4 and MVR-ΔIR47 per cell. At 48 hours post infection, the morphology of each cell was observed by EVOS XL core microscopy and raw digital images were documented.

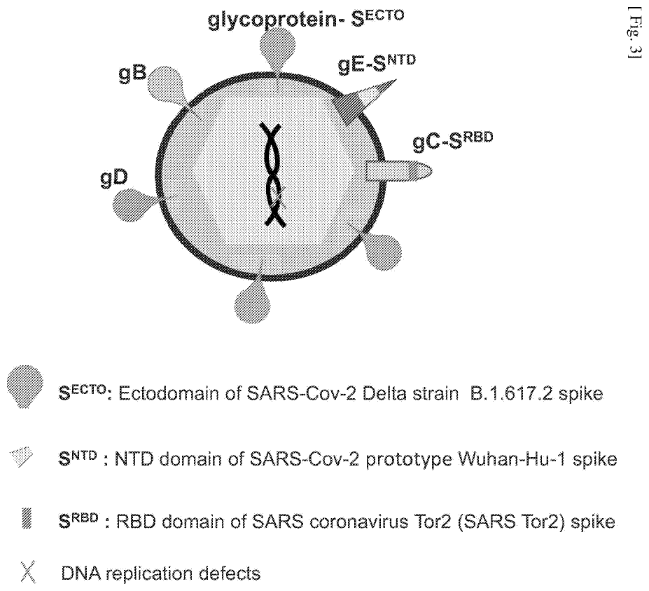

As shown in FIG. 2, replication-defective MVR-ΔIR4 and MVR-ΔIR47 viruses can only replicate and cause cytopathic effect (CPE) in E5 cells (Vero-derived, ICP4 complementing cell line) rather than in Vero cells.

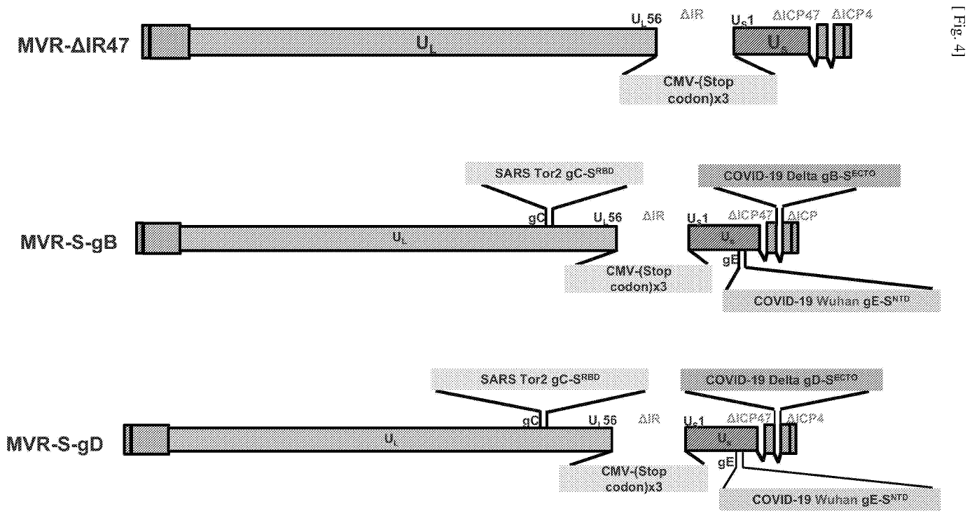

FIG. 3 shows construction model of non-replicating HSV-1 based sarbecovirus spike protein vaccine virus.

The green bulb shape represents ectodomain of SARS-Cov-2 Delta strain B.1.617.2 spike (S^{ECTO}). The glycoprotein-S^{ECTO} is a chimera, in which the S^{ECTO} was connected to the transmembrane domain (TM) and cytoplasmic tail (CT) of the gB or gD proteins. The light green trapezoid represents NTD domain of SARS-Cov-2 wild-type Wuhan-Hu-1 spike (S^{NTD}). The gE-S^{NTD} is a chimera, in which the gE protein amino acids from 237-382 was replaced by the NTD domain of SARS-Cov-2 WT Wuhan-Hu-1 spike. The red rectangle represents RBD domain of SARS coronavirus Tor2 (SARS Tor2) spike (S^{RBD}). The gC-S^{RBD} is a chimera, in which the gC protein amino acids from 275-367 was replaced by the RBD domain of SARS coronavirus Tor2 (SARS Tor2) spike. The red cross represents the viral genome replication defective modification.

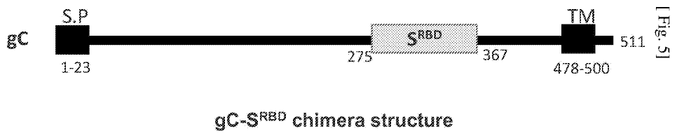

FIG. 4 shows schematic diagram of the MVR-ΔIR47, MVR-S-gB or MVR-S-gD genomes.

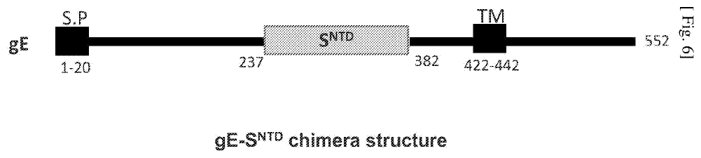

FIG. 5 shows the structure of gC-S^{RBD} chimera, in which the gC protein amino acids from 275-367 was replaced by the RBD domain of SARS coronavirus Tor2 (SARS Tor2) spike.

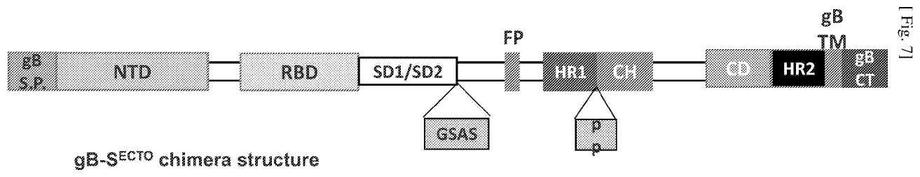

FIG. 6 shows the structure of gE-S^{NTD} chimera, in which the gE protein amino acids from 237-382 was replaced by the NTD domain of SARS-Cov-2 WT Wuhan-Hu-1 spike.

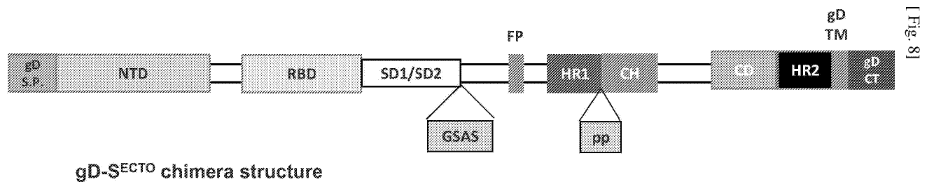

FIG. 7 shows the structure of gB-S^{ECTO} chimera, in which the S^{ECTO} was connected to the transmembrane domain and cytoplasmic tail (TM/CT) of the gB protein.

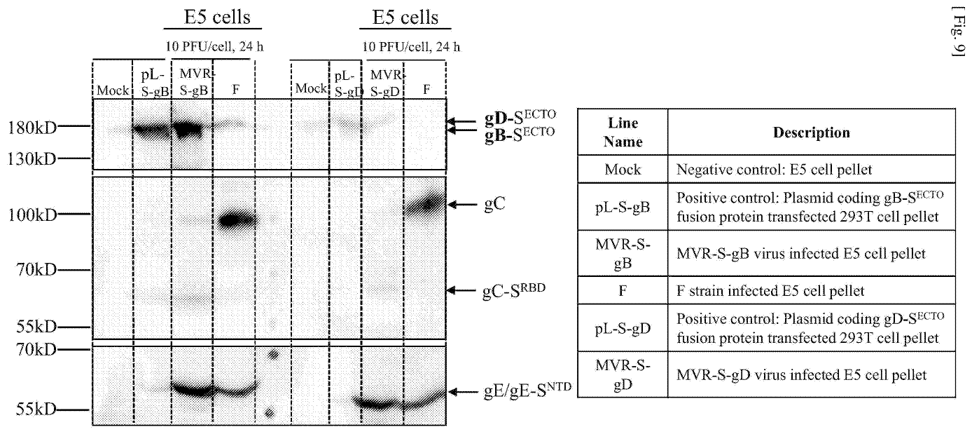

FIG. 8 shows the structure of gD-S^{ECTO} chimera, in which in which the S^{ECTO} was connected to the transmembrane domain and cytoplasmic tail (TM/CT) of the gD protein

### Protein Expression of Non-Replicating HSV-1 Based Spike Protein Vaccine Virus in E5 Cells

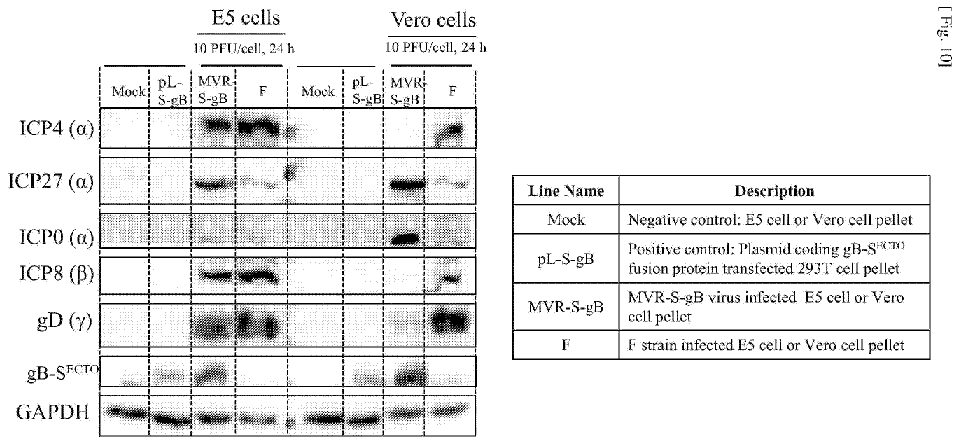

FIG. 9 shows the accumulation of gC, gC-S^{RBD}, gE-S^{NTD}, gB-S^{ECTO} or gD-ECTO proteins in MVR-S-gB and MVR-S-gD infected E5 cells. 75 cm² flask were seeded with 6×10⁶ of E5 or Vero cells. After overnight incubation at 37° C., cells were mock infected or infected at 10 PFU of HSV-1 (F), MVR-S-gB, MVR-S-gD per cell. The cells were harvested at 24 hours post infection. The gC, gC-S^{RBD}, gE-S^{NTD}, gB-S^{ECTO} or gD-S^{ECTO} protein expressions were detected by immunoblotting assay.

As shown in FIG. 9, spike fusion protein is more efficiently expressed by MVR-S-gB virus than MVR-S-gD virus in infected E5 cells.

### HSV Viral Protein Accumulation in MVR-S-gB Virus Infected E5 and Vero Cells

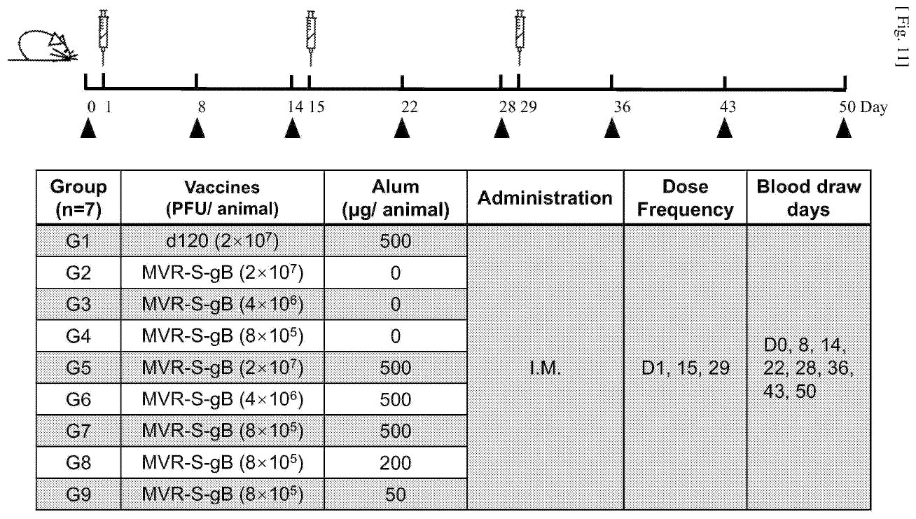

FIG. 10 shows the accumulation of HSV-1 representative viral proteins in MVR-S-gB virus infected E5 and Vero cells. 75 cm² flask were seeded with 6×10⁶ of E5 or Vero cells. After overnight incubation at 37° C., cells were mock infected and infected at 10 PFU of HSV-1 (F), MVR-S-gB per cell. The cells were harvested at 24 hours post infection. The expression of the representative viral proteins ICP4, ICP27, ICP0, ICP8, gD and MVR-S-gB virus fusion protein gB-S^{ECTO} were detected by immunoblotting assay.

As shown in FIG. 10, MVR-S-gB efficiently expressed all detected viral proteins, ICP4(α), ICP27(α), ICP0(α), ICP8(β) and gD(γ), representing viral immediate early (α), early (β), and late (γ) genes in ICP4 complementing E5 cells (Vero-derived, ICP4 complementing cell line), while only expressed immediate early (a) gene products ICP27 and ICP0 in Vero cell. All these results indicated MVR-S-gB virus with the IR region and ICP4 gene in TR region deletion which leading both copies of ICP4 gene disruption make the vector replication-defective and be a promising sarbecovirus vaccine candidate.

### Animal Vaccination

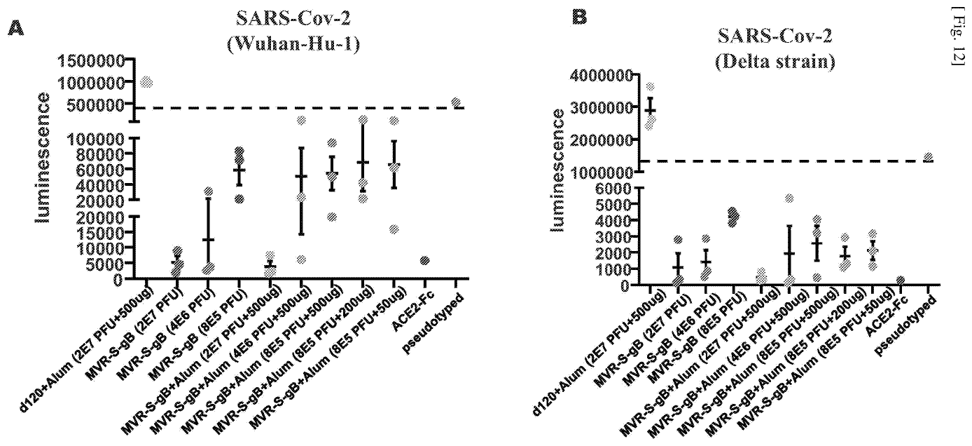

FIG. 11 shows the design of animal immunization. Six-to-eight-week-old female BalB/c mice were used. Group 1 (n=7) were vaccinated with negative control d120 virus which lacking ICP4 genes in the presence of aluminum (Alum). Groups 2~9 (n=7) were vaccinated with MVR-S-gB in the absence or presence of Alum. The d120 or MVR-S-gB was administered via the intramuscular (I.M.) route at D1, D15 and D29. Serum samples were collected at DO, D8, D14, D22, D28, D36, D43 and D50.

Six-to-eight-week-old female BALB/c mice were either vaccinated with MVR-S-gB (n=7) or negative control d120 virus in the absence or presence of Alum. Three immunizations were performed via the intramuscular route at D1, D15 and D29. Serum samples collected at D36 were used for pseudotyped virus neutralization assays. Three out of seven mouse serum sample were tested in neutralization assay using HIV-based Wuhan-Hu-1 strain or B.1.617.2 Delta strain pseudovirus in technical duplicate. Pooled sera from each group were tested in neutralization assay using Omicron BA1 strain pseudovirus in technical duplicate.

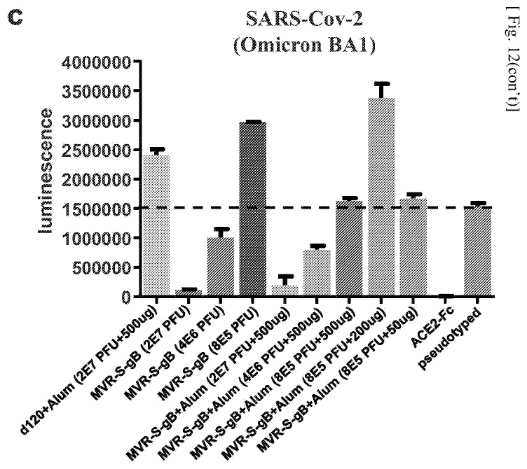

FIG. 12 shows the microneutralization assays using HIV-based pseudotyped virus. As shown in FIG. 12, all the groups vaccinated with MVR-S-gB induced neutralization antibodies against Wuhan-Hu-1 strain (A) and Delta strain (B) in a dose-dependent manner. Furthermore, the groups immunized with 4×10⁶ PFU or 2×10⁷ PFU of MVR-S-gB induced neutralization antibodies against Omicron BA1 strain (C) and also in a dose-dependent manner, which indicated cross-neutralizing antibodies against pan-sarbecovirus were induced by the MVR-S-gB.

MVR-S-gB vaccines can induce long-lasting neutralizing antibodies up to at least 6 months.

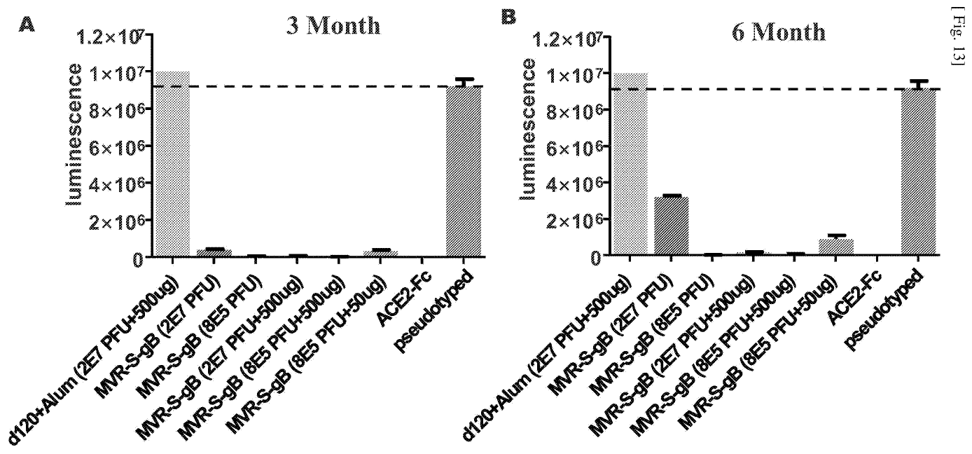

Six-to-eight-week-old female BALB/c mice were either vaccinated with MVR-S-gB (n=7) or negative control d120 virus in the absence or presence of Alum. Three immunizations were performed via the intramuscular route at D1, D15 and D29. Serum samples were collected at 3 months (FIG. 13A) and 6 months (FIG. 13B) after third vaccination and used for pseudotyped virus neutralization assays. Pooled mouse sera from each group were tested in a pseudovirus neutralization assay against Delta strain in technical duplicate.

As shown in FIG. 13, the groups vaccinated with MVR-S-gB can induced long-lasting neutralization antibodies against Delta strain up to at least 6 months.

1. A replication incompetent Herpes Simplex Virus type 1 (HSV-1) viral vaccine, comprising a modified genome of HSV-1 and at least one antigen, wherein the modification comprises
a deletion of an internal inverted repeat region which causes deletions of one copy of each of double-copy genes including ICP0, ICP34.5, ICP4 and latency-associated transcript (LAT),
an inactivating mutation in ICP47, and
an inactivating mutation in the other copy of ICP4 in a terminal repeat, and wherein
a first antigen of the at least one antigen is driven by a promoter of an immediate early gene of wild-type HSV-1.

2. The replication incompetent HSV-1 viral vaccine of claim 1, wherein the immediate early gene of wild-type HSV-1 is ICP0, ICP27, ICP4, ICP22 or ICP47.

3. The replication incompetent HSV-1 viral vaccine of claim 1, wherein the immediate early gene of wild-type HSV-1 is ICP4.

4. The replication incompetent HSV-1 viral vaccine of claim 3, wherein the inactivating mutation in the other copy of ICP4 in the terminal repeat is a deletion in a coding sequence of ICP4.

5. The replication incompetent HSV-1 viral vaccine of claim 4, wherein the first antigen of the at least one antigen is operably linked to the promoter of ICP4 in the terminal repeat.

6. The replication incompetent HSV-1 viral vaccine of claim 1, wherein the first antigen of the at least one antigen together with the driving promoter is inserted into a position corresponds to the deleted internal inverted repeat region.

7. The replication incompetent HSV-1 viral vaccine of claim of claim 1, wherein a second antigen of the at least one antigen is fused into a first HSV-1 glycoprotein.

8. The replication incompetent HSV-1 viral vaccine of claim 7, wherein a third antigen of the at least one antigen is fused into a second HSV-1 glycoprotein.

9. The replication incompetent HSV-1 viral vaccine of claim 8, wherein the first or second HSV-1 glycoprotein is glycoprotein gC or gE.

10. The replication incompetent HSV-1 viral vaccine of claim 9, wherein the glycoprotein gC is altered to inactivate C3 binding, and wherein the glycoprotein gE is altered to inactivate FcR binding.

11. The replication incompetent HSV-1 viral vaccine of claim 10, wherein the glycoprotein gC contains a deletion in C3 binding domain, and wherein the glycoprotein gE contains a deletion in FcR binding domain.

12. The replication incompetent HSV-1 viral vaccine of claim 1, wherein the first antigen is linked to a glycoprotein gB or gD signal peptide at N terminus, and to a transmembrane-intravirion domain of glycoprotein gB or gD at C terminus.

13. The replication incompetent HSV-1 viral vaccine of claim 8, wherein the first, second or third antigen is from a virus, a bacterium or a parasite.

14. The replication incompetent HSV-1 viral vaccine of claim 8, wherein the first, second and third antigen are from sarbecovirus.

15. The replication incompetent HSV-1 viral vaccine of claim 14, wherein the first, second and third antigen are from SARS-Cov, SARS-Cov-2 and variants thereof.

16. The replication incompetent HSV-1 viral vaccine of claim 15, wherein the first antigen is from delta or omicron variant of SARS-Cov-2, the second and third antigens are from SARS-Cov, SARS-Cov-2 and variants thereof.

17. The replication incompetent HSV-1 viral vaccine of claim 16, wherein the first antigen is from delta variant of SARS-Cov-2, the second antigen is from SARS-Cov Tor2 strain, and the third antigen is from SARS-Cov-2 Wuhan-Hu-1 strain.

18. The replication incompetent HSV-1 viral vaccine of claim 8, wherein the first antigen is an ectodomain of Spike glycoprotein of delta variant of SARS-Cov-2 or an immunogenically equivalent variant thereof.

19. The replication incompetent HSV-1 viral vaccine of claim 18, wherein the ectodomain or the immunogenically equivalent variant thereof is linked to a glycoprotein gB signal peptide at N terminus, and to a transmembrane-intravirion domain of glycoprotein gB at C terminus.

20. The replication incompetent HSV-1 viral vaccine of claim 19, wherein the immunogenically equivalent variant of the ectodomain has a K986P/V987P mutation and/or a 682-GSAS-685 mutation.

21. The replication incompetent HSV-1 viral vaccine of claim 18, wherein one of the second and third antigen is a receptor binding domain of Spike glycoprotein of SARS-Cov Tor2 strain, and the other is an N-terminal domain of Spike glycoprotein of SARS-Cov-2 Wuhan-Hu-1 strain, or an immunogenically equivalent variant each thereof.

22. The replication incompetent HSV-1 viral vaccine of claim 21, wherein the second antigen is a receptor binding domain of Spike glycoprotein of SARS-Cov Tor2 strain or an immunogenically equivalent variant thereof and the first HSV-1 glycoprotein is glycoprotein gC.

23. The replication incompetent HSV-1 viral vaccine of claim 22, wherein the receptor binding domain or an immunogenically equivalent variant thereof is fused into glycoprotein gC in replace of its C3 binding domain.

24. The replication incompetent HSV-1 viral vaccine of claim 21, wherein the second antigen is an N-terminal domain of Spike glycoprotein of SARS-Cov-2 Wuhan-Hu-1 strain or an immunogenically equivalent variant thereof and the first HSV-1 glycoprotein is glycoprotein gE.

25. The replication incompetent HSV-1 viral vaccine of claim 24, wherein N-terminal domain of Spike glycoprotein of SARS-Cov-2 Wuhan-Hu-1 strain or an immunogenically equivalent variant thereof is fused into glycoprotein gE in replace of its FcR binding domain.

26. The replication incompetent HSV-1 viral vaccine of claim 1, wherein the inactivating mutation in ICP47 is a deletion in a coding sequence of ICP47.

27. The replication incompetent HSV-1 viral vaccine of claim 5, wherein the inverted internal repeat region is replaced by a CMV promoter followed by three repeats of stop codon.

28. The replication incompetent HSV-1 viral vaccine of claim 1, wherein the modified genome contains one copy of ICP0, LAT and ICP34.5, UL1 to UL56, and US1 to US11.

29. A vaccine composition, comprising a replication incompetent HSV-1 viral vaccine of claim 1, and a pharmaceutically acceptable carrier.

30. The vaccine composition of claim 29, wherein the pharmaceutically acceptable carrier comprises an adjuvant.
